(19) United States
(12) Patent Application Publication
(10) Pub. No.: US 2024/0294592 A1
(43) Pub. Date: Sep. 5, 2024

(54) A 5'-CAP ANALOG MODIFIED mRNA

(71) Applicant: Nanjing GeneLeap Biotechnology Co., Ltd., Nanjing (CN)

(72) Inventors: Dan Tse, Andover, MA (US); Teodelinda Mirabella, Arlington, MA (US); Dehua Wang, Nanjing (CN); Sheng Bi, Nanjing (CN); Lakshmi Bhagat, Framingham, MA (US)

(21) Appl. No.: 18/170,512

(22) Filed: Feb. 16, 2023

(51) Int. Cl. C07K 14/54

(52) U.S. Cl. CPC C07K 14/5434

(57) ABSTRACT

The invention provides mRNAs modified with a series of novel 5′-cap analogs, wherein the 5′-cap analogs can improve transcription, mRNA stability, and mRNA translation efficiency and durability, and the mRNA modified with the 5′-cap analogs could inhibit tumor growth and improve survival significantly better than the control CleanCap modified-mRNA.

### TECHNICAL FIELD

This invention is directed to modification of the 5′-cap structure of RNA using nucleic acid complementary to the 5′ end of a messenger RNA to effect alteration of the 5′-cap structure of the RNA, thereby modulating its function. Thus, the invention generally relates to gene expression in animal cells, and to protein expression in particular.

### BACKGROUND

RNA degradation is a key process in the regulation of gene expression. Any functional mRNA, including mRNA therapeutics, must be protected from the RNA degradation machinery. Yet, the protection found in nature is temporary, and could be improved by suitable chemical modifications. In recent years, in vitro transcribed (IVT) mRNA has emerged as a promising route for therapeutic gene synthesis. Since proper mRNA function is dependent on its structural regulatory elements, including 5′-cap and polyA modifications at the 3′-end of an mRNA, these elements play a critical role in mRNA stability. Therefore, rational modifications of 5′-cap structure can improve the potency of mRNA therapeutics through improving RNA stability and facilitating its cellular and in vivo translation (Nature Reviews Drug Discovery, Vol. 17, 261).

In eukaryotes, 7-methylguanosine 5′-cap on mRNA is crucial for efficient recognition by protein synthesis machinery, e.g. ribosomes, in vitro and in vivo. Among mRNA manufacturing processes, a co-transcriptional method is more favored for in vitro transcription than a post-transcriptional 5′-cap method due to its simplicity and efficiency (Genes and Development, Vol. 20, 1838). To be suitable for co-transcription, 7-methylguanosine containing di- and trinucleotides with 5′-5′-triphosphate linker, known as cap-0, cap-1, ARCA and CleanCap have been developed and are currently used in approved mRNA COVID-19 vaccines and other multiple mRNAs that are currently in clinical trials (Nature, Vol. 596, 109). Published literature on 5′-cap analogs mainly rely on 7-methylguanosine (7 mG) and its 2′- and 3′-substituents (U.S. Pat. No. 10,925,935B1, U.S. Pat. No. 9,295,717B2), phosphate replacement in the inter-nucleotide linkers (U.S. Pat. No. 10,570,388B2), such as substitution of a non-bridging oxygen with a sulphur moiety to give a phosphorothioate linkage (U.S. Pat. No. 8,153,773B2), or extended nucleotides such as in CleanCap® (U.S. Pat. No. 10,519,189B2). However, there is a need for i) improving capping efficiency in IVT, ii) increasing resistance against decapping machinery, and iii) improving binding to T7 or other polymerases, via using non-natural nucleoside modifications to replace 7 mG or triphosphate modifications that have not been explored.

Therefore, we designed, synthesized, and evaluated a series of novel 5′-cap structures for mRNA therapeutic programs for a number of properties: stability, transcription and/or translation efficiency and durability, and modified the mRNA with said 5′-cap structures.

### BRIEF SUMMARY

The present disclosure provides mRNA modified with a series of novel 5′-cap analogs. The 5′-cap analogs of the present disclosure can improve transcription, mRNA stability, and mRNA translation efficiency and durability, and the mRNA modified with the present 5′-cap analogs could inhibit tumor growth and improve survival significantly better than the control CleanCap modified-mRNA.

In one aspect, the present disclosure provides an mRNA modified with a 5′-cap analog, comprising the 5′-cap analog of formula (I) below or a stereoisomer, tautomer or salt thereof:

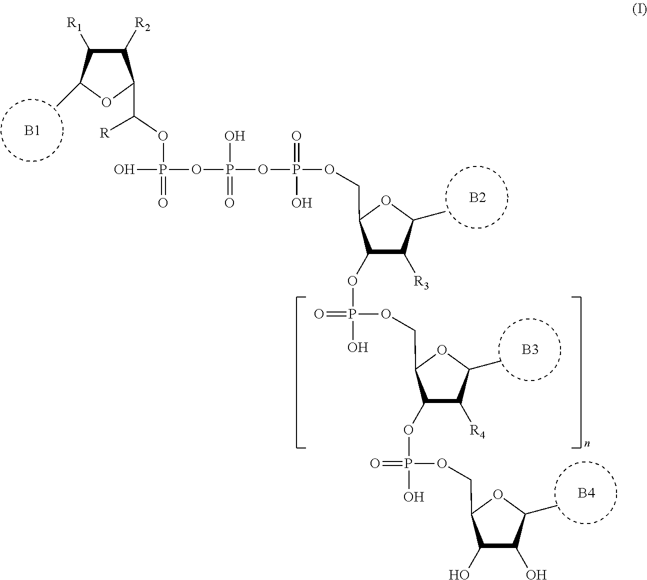

wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl or C₂₋₆ alkynyl is optionally substituted with one or more of halogen, OH, and/or C₁₋₆ alkoxyl; and
n is an integer selected from 0-1,
wherein said mRNA comprises, from 5′ to 3′, an interleukin 12 subunit β (IL12B) polypeptide coding sequence and an interleukin 12 subunit α (IL12A) polypeptide coding sequence.

In another aspect, the present disclosure provides methods of synthesizing the 5′-cap analog of formula (I).

In still another aspect, the present disclosure provides methods of synthesizing an RNA molecule (e.g., mRNA) in vitro. The method can include reacting unmodified or modified ATP, unmodified or modified CTP, unmodified or modified UTP, unmodified or modified GTP, a 5′-cap analog of formula (I) or a stereoisomer, tautomer or salt thereof, and a polynucleotide template, in the presence of an RNA polymerase, under a condition conducive to transcription by the RNA polymerase of the polynucleotide template into one or more RNA copies, whereby at least some of the RNA copies incorporate the 5′-cap analog of formula (I) or a stereoisomer, tautomer or salt thereof to make an RNA molecule (e.g., mRNA).

In another aspect, the present disclosure provides a composition or a kit comprising the mRNA modified with a 5′-cap analog of the present disclosure.

Further, the compounds or methods described herein can be used for research (e.g., studying interaction of an in vitro RNA transcript with certain enzymes) or other non-therapeutic purposes.

### BRIEF DESCRIPTION OF THE SEVERAL VIEWS OF THE DRAWINGS

FIG. 1 provides the expression of IL-12 protein in Hela cells at 24 hr after transfection with hGL-001 mRNA co-transcriptionally capped with GL-Cap 9, or hMD-001 mRNA co-transcriptionally capped with CleanCap®.

FIG. 2 provides the expression of IL-12 protein in HeLa cells at 48 hr after transfection with hGL-001 mRNA co-transcriptionally capped with GL-Cap 9 or GL-Cap 5, and with hMD-001 mRNA co-transcriptionally capped with CleanCap®.

FIGS. 3A and 3B show the survival for the synthesized GL-Cap 5 capped IL-12 mRNA with dose titration on MC38 Tumor Model. In FIG. 3A, data is presented as mean±SEM, and in FIG. 3B, data is presented as Kaplan-Meier curves.

FIGS. 4A and 4B show the survival for the synthesized GL-Cap 9 capped IL-12 mRNA with dose titration on MC38 Tumor Model. In FIG. 4A, data is presented as mean±SEM, and in FIG. 4B, data is presented as Kaplan-Meier curves.

### DETAILED DESCRIPTION

mRNA consists of an open reading frame (ORF) flanked by the 5′- and 3′-untranslated region (5′UTR, 3′UTR), a poly-adenosine monophosphate tail (poly A) and an inverted N7-methylguanosine-containing cap structure. It is both chemically and enzymatically less stable than the corresponding DNA, hence the protein production subsequent to the ribosomal recruitment of the mRNA is temporary. In addition, the mRNA must be present in a so-called “closed loop” conformation in order to produce the target protein. While part of the active closed-loop conformation, the mRNA makes contact with the ribosomal machinery through the cap that binds to the eukaryotic initiation factor 4E (eIF4E) and the polyA tail attached through the poly A-binding protein (PABP). The eIF4E and PABP are connected through a skeletal protein eIF4G closing the active loop. Disruption of the mRNA circularized form leads to cessation of protein production and eventually enzymatic degradation of the mRNA itself chiefly by action of the de-capping enzyme system DCP1/2 and or through a poly-A ribonuclease (PARN) mediated de-adenylation. See, e.g., Richard J. Jackson et al., “The mechanism of eukaryotic translation initiation and principles of its regulation”, Molecular Cell Biology, vol. 110, 113-127, 2010.

The cap structure is a crucial feature of all eukaryotic mRNAs. It is recognized by the ribosomal complex through the eukaryotic initiation factor 4E (eIF4E). mRNAs lacking the 5′-cap terminus are not recognized by the translational machinery and are incapable of producing the target protein (see, e.g., Colin Echeverria Aitken, Jon R Lorsch: “A mechanistic overview of translation initiation in eukaryotes”, Nature Structural and Molecular Biology, vol. 16, no. 6, 568-576, 2012.)

The crude messenger RNA produced during the transcription process (“primary transcript”) is terminated by a 5′-triphosphate, which is converted to the respective 5′-diphosphate by the action of the enzyme RNA-triphosphatase. Then a guanylyl-transferase attaches the terminal inverted guanosine monophosphate to the 5′-terminus, and an N7MTase-mediated N7-methylation of the terminal, inverted guanosine completes the capping process.

The wild-type 5′-cap structure is vulnerable to enzymatic degradation, which is part of the regulation mechanism controlling protein expression. According to this mechanism, the enzymatic system DCP1/2 performs a pyrophosphate hydrolysis between the second and the third phosphate groups of the cap structure, removing the N7-methylated guanosine diphosphate moiety leaving behind an mRNA terminated in a 5′-monophosphate group. This in turn is quite vulnerable to exonuclease cleavage and will lead to rapid decay of the remaining oligomer. See, e.g., R. Parker, H. Song: “The Enzymes and Control of Eukaryotic Turnover”, Nature Structural & Molecular Biology, vol. 11, 121-127, 2004.

The present disclosure provides a series of novel mRNA 5′-cap analogs, and synthetic methods for making these cap analogs, and further provides mRNA molecules modified with the cap analogs disclosed herein which impart properties that are advantageous to therapeutic development.

In one aspect, the present disclosure provides an mRNA modified with 5′-cap analog, comprising the 5′-cap analog of formula (I) below or a stereoisomer, tautomer or salt thereof:

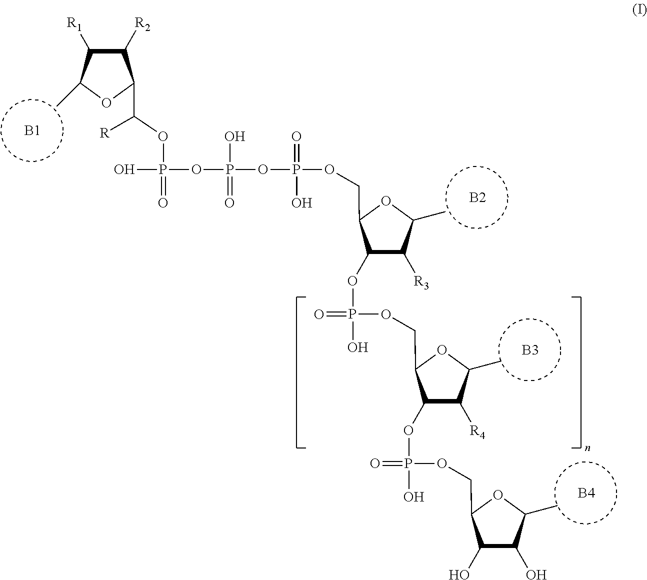

wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl; and
n is an integer selected from 0-1,
wherein said mRNA comprises, from 5′ to 3′, an interleukin 12 subunit β (IL12B) polypeptide coding sequence and an interleukin 12 subunit α (IL12A) polypeptide coding sequence.

In certain embodiments, ring B1 and B4 are each independently guanine (G), and ring B2 and B3 are each independently adenine (A).

In certain embodiments, R is independently selected from H and methyl.

In certain embodiments, R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

In certain embodiments, R₁ and R₂ are each independently selected from OH, —O(CH₂)₂OCH₃ and F.

In certain embodiments, R₃ is independently selected from methoxy, F and LNA.

In certain embodiments, R₄ is independently selected from F and LNA.

In another aspect, the present disclosure provides an mRNA modified with 5′-cap analog, comprising the 5′-cap analog of formula (IA) below or a stereoisomer, tautomer or salt thereof:

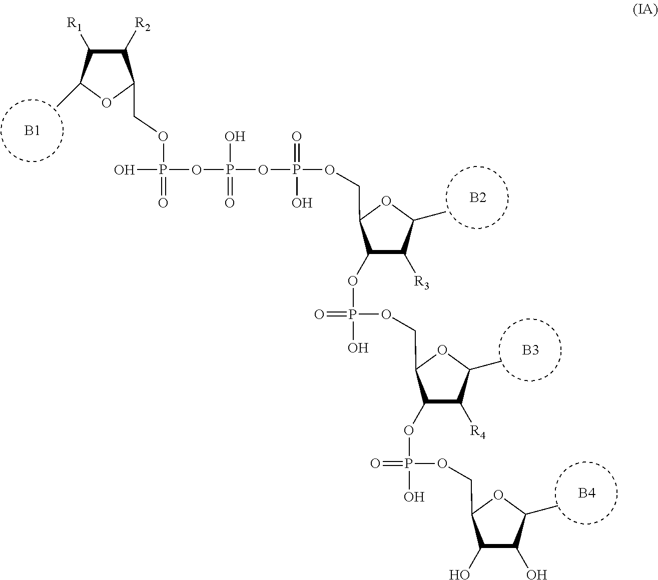

wherein:
ring B1, B2, B3 and B4 are each independently selected from a nucleobase and a modified nucleobase; and
R₃ and R₄ are each independently selected from halogen and LNA,
wherein said mRNA comprises, from 5′ to 3′, an interleukin 12 subunit β (IL12B) polypeptide coding sequence and an interleukin 12 subunit α (IL12A) polypeptide coding sequence.

In certain embodiments, ring B1 and B4 are each independently guanine (G), and ring B2 and B3 are each independently adenine (A).

In certain embodiments, R₃ and R₄ are each independently selected from F and LNA.

In another aspect, the present disclosure provides an mRNA modified with 5′-cap analog, comprising the 5′-cap analog of formula (IB) below or a stereoisomer, tautomer or salt thereof:

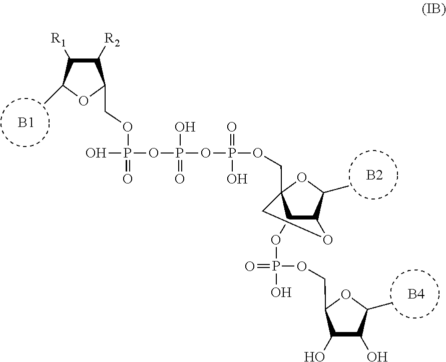

wherein:
ring B1, B2 and B4 are each independently selected from a nucleobase and a modified nucleobase;
R₁ and R₂ are each independently selected from OR₅ and halogen; and
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl,
wherein said mRNA comprises, from 5′ to 3′, an interleukin 12 subunit β (IL12B) polypeptide coding sequence and an interleukin 12 subunit α (IL12A) polypeptide coding sequence.

In certain embodiments, ring B1 and B4 are each independently guanine (G), and ring B2 is adenine (A).

In certain embodiments, R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

In certain embodiments, R₁ and R₂ are each independently selected from OH, —O(CH₂)₂OCH₃ and F.

In another aspect, the present disclosure provides an mRNA modified with 5′-cap analog, comprising the 5′-cap analog selected from any of the following structures or a stereoisomer, tautomer or salt thereof:

| No. | Chemical name | Chemical structure |
|---|---|---|
| GL-Cap 1 | 7mGppp(locked)Ap(locked)ApG | 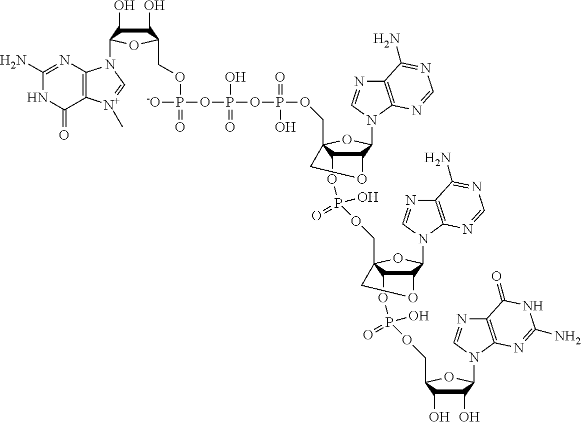 |
| GL-Cap 2 | 7mGppp(2-′F)Ap(2′-F)ApG | 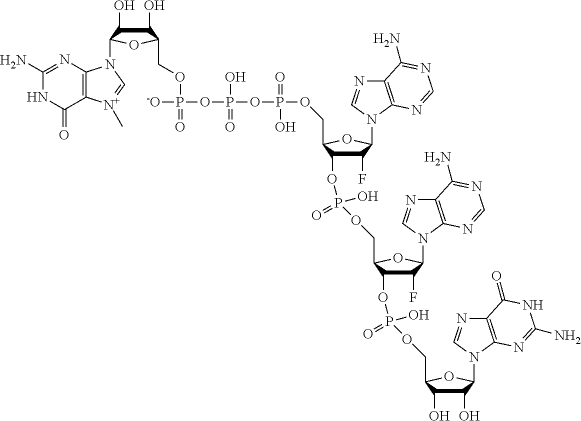 |
| GL-Cap 3 | 7mGppp(2′-F)Ap(locked)ApG | 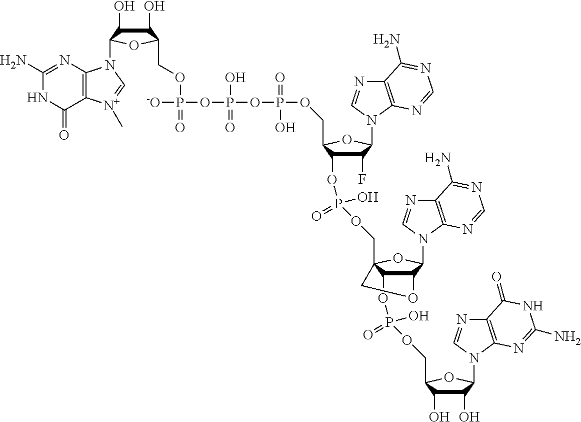 |
| GL-Cap 4 | 7mGppp(locked)Ap(2′-F)ApG | 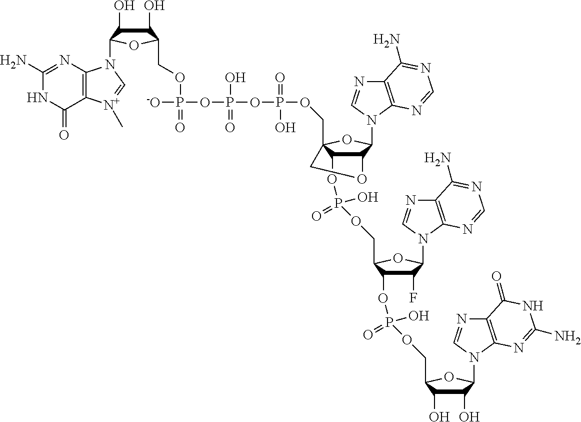 |
| GL-Cap 5 | 7mGppp(locked)ApG | 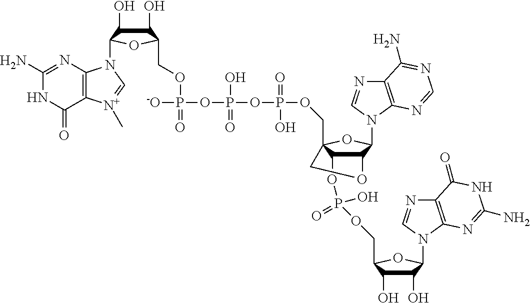 |
| GL-Cap 6 | 7m-3′-MOEGppp(locked)ApG | 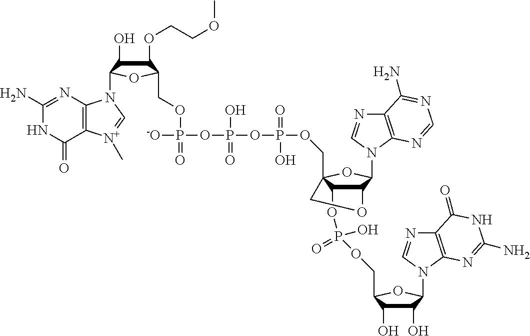 |
| GL-Cap 7 | 7m-2′-FGppp(locked)ApG | 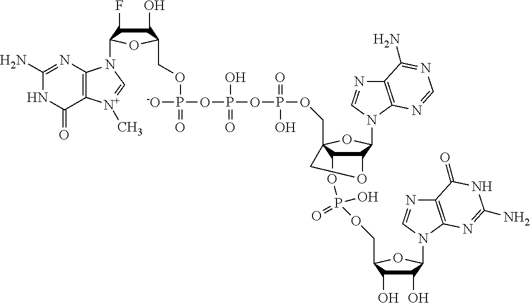 |
| GL-Cap 8 | 7m-2′-MOEGppp(locked)ApG | 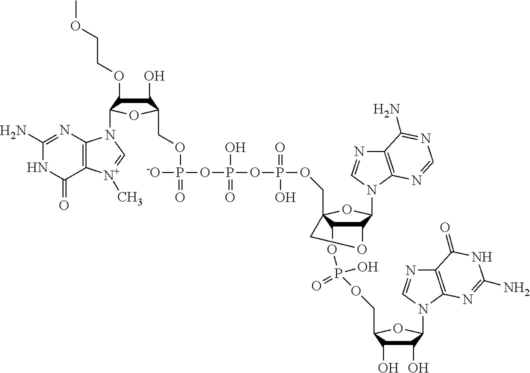 |
| GL-Cap 9 | 7m-3′-MOE-5′-RmGppp(2′- Ome)ApG | 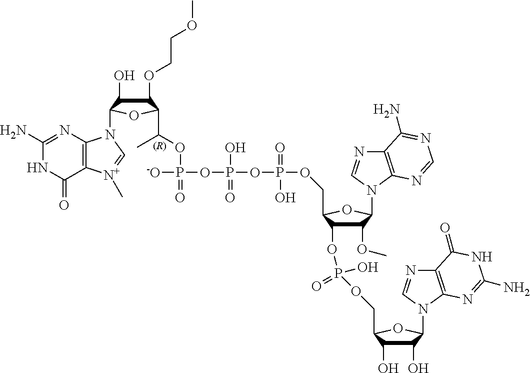 |

wherein said mRNA comprises, from 5′ to 3′, an interleukin 12 subunit β (IL12B) polypeptide coding sequence and an interleukin 12 subunit α (IL12A) polypeptide coding sequence.

In certain embodiments, the IL12B and IL12A of the present disclosure are human IL12B and human IL12A, or the IL12B and IL12A of the present disclosure are mice IL12B and mice IL12A.

In certain embodiments, the IL12B polypeptide coding sequence of the present disclosure comprises SEQ ID NO:2 or SEQ ID NO:9, and/or the IL12A polypeptide coding sequence of the present disclosure comprises SEQ ID NO:3 or SEQ ID NO:10.

In certain embodiments, the mRNA of the present disclosure further comprises a linker coding sequence linking IL12B polypeptide and IL12A polypeptide, wherein preferably said linker coding sequence comprises SEQ ID NO:4 or SEQ ID NO:11.

In certain embodiments, the mRNA of the present disclosure further comprises 5′UTR and/or 3′UTR, wherein preferably said 5′UTR comprises SEQ ID NO:5, and/or said 3′UTR comprises SEQ ID NO:7.

In certain embodiments, the mRNA of the present disclosure further comprises a promoter, wherein preferably said promoter comprises SEQ ID NO:6 or SEQ ID NO:12.

In certain embodiments, the mRNA of the present disclosure comprises SEQ ID NO:1 or SEQ ID NO:8.

In another aspect, the present disclosure provides a composition, preferably a pharmaceutical composition comprising the mRNA modified with 5′-cap analog of the present disclosure. The composition, in particular pharmaceutical composition, of this aspect may comprise the mRNA modified with 5′-cap analog of the present disclosure in combination with one or more pharmaceutically acceptable excipients. In certain embodiments, the pharmaceutical composition comprises an mRNA modified with 5′-cap analog of the present disclosure, one or more pharmaceutically acceptable excipients and one or more additional/supplementary active compounds. In certain embodiments, the composition of the present disclosure is a pharmaceutical composition, preferably a lipid nanoparticle (LNP).

In another aspect, the present disclosure provides a kit comprising the mRNA modified with 5′-cap analog of the present disclosure. In certain embodiments, the kit may further comprise reagents typically used in in vitro transcription reactions (e.g., NTPs, an RNA polymerase, one or more buffers, and/or a DNA template) and/or instructions for use.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

### Definitions

For the purpose of the current disclosure, the following definitions shall in their entireties be used to define technical terms, and shall also, in their entireties, be used to define the scope of the composition of matter for which protection is sought in the claims.

As used herein, the term “LNA” or “locked nucleic acid” refers to a methylene bridge between the 2′O and 4′C of the nucleotide monomer or to a sugar analog, a nucleoside, a nucleotide monomer, or a nucleic acid, each of which contains such bridge. For example,

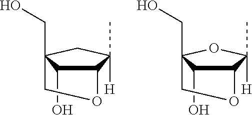

or those described in WO99/14226 and Kore et al, J. AM. CHEM. SOC. 2009, 131, 6364-6365, the contents of each of which are incorporated herein by reference in their entireties.

As used herein, the term “nucleobase” refers to a nitrogen-containing heterocyclic moiety, which are the parts of the nucleic acids that are involved in the hydrogen-bonding that binds one nucleic acid strand to another complementary strand in a sequence-specific manner. The most common naturally-occurring nucleobases are: adenine (A), cytosine (C), guanine (G), thymine (T), and uracil (U).

The term “modified nucleobase” refers to a moiety that can replace a nucleobase. The modified nucleobase mimics the spatial arrangement, electronic properties, or some other physicochemical property of the nucleobase and retains the property of hydrogen-bonding that binds one nucleic acid strand to another in a sequence-specific manner. A modified nucleobase can pair with at least one of the five naturally occurring bases (uracil, thymine, adenine, cytosine, or guanine) without substantially affecting the melting behavior, recognition by intracellular enzymes, or activity of the oligonucleotide duplex. The term “modified nucleoside” or “modified nucleotide” refers to a nucleoside or nucleotide that contains a modified nucleobase and/or other chemical modification(s) disclosed herein, such as modified sugar, modified phosphorus atom bridges or modified internucleoside linkage.

Non-limiting examples of suitable nucleobases include, but are not limited to, hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, and dihydrouridine.

As used herein, “halo” or “halogen” refers to fluoro, chloro, bromo, and iodo.

The term “alkyl” and “C₁₋₆-alkyl” are intended to mean a linear or branched saturated hydrocarbon chain wherein the longest chain has from one to six carbon atoms, such as methyl, ethyl, n-propyl, i-propyl, n-butyl, s-butyl, t-butyl, n-pentyl, s-pentyl, and n-hexyl.

The term “alkenyl” and “C₁₋₆ alkenyl” are intended to mean a linear or branched unsaturated hydrocarbon chain wherein the longest chain has from two to six carbon atoms, such as ethenyl, propenyl, butenyl, pentenyl, hexenyl, and branched alkenyl groups.

The term “alkynyl” and “C₁₋₆ alkynyl” are intended to mean a linear or branched unsaturated hydrocarbon chain which contains at least one triple bond, wherein the longest chain has from two to six carbon atoms, such as ethynyl, propynyl, butynyl, pentynyl, hexynyl, and branched alkynyl groups.

The term “alkoxy” and “C₁₋₆ alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy, pentoxy and hexyloxy groups.

As used herein, “carbocycle”, “cycloalkyl” and “carbocyclic ring” is intended to include any stable monocyclic or bicyclic ring having the specified number of carbons, any of which may be saturated or unsaturated. For example, C₃₋₈ cycloalkyl is intended to include a monocyclic or bicyclic ring having 3, 4, 5, 6, 7 or 8 carbon atoms. Examples of carbocycles include, but are not limited to, cyclopropyl, cyclobutyl, cyclobutenyl, cyclopentyl, cyclopentenyl, and cyclohexyl.

As used herein, “heterocycle”, “heterocycloalkyl” or “heterocyclic group” includes any ring structure (saturated or unsaturated) which contains at least one ring heteroatom (e.g., N, O or S). Heterocycle includes heterocycloalkyl. Examples of heterocycloalkyl include, but are not limited to, morpholine, pyrrolidine, tetrahydrothiophene, piperidine, piperazine, oxetane, pyran, tetrahydropyran, azetidine, and tetrahydrofuran.

The term “amine protecting group” refers to a protecting group for amines. Examples of amine protecting groups include but are not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, Tosyl (Ts). See also Chem. Rev. 2009, 109, 2455-2504 for additional amine protecting groups, the contents of which are incorporated herein by reference in their entirety.

The term “stereoisomer” refers to a compound which is composed of the same atoms, bonded by the same bonds, but which has a different three-dimensional structure. The present invention will cover various stereoisomers and mixtures thereof.

The term “tautomer” refers to the isomer formed by transferring a proton from one atom of a molecule to another atom of the same molecule. All tautomeric forms of the compounds of the present invention will also be included within the scope of the present invention.

The compounds of the present invention may contain one or more chiral carbon atoms, and thus may produce an enantiomer, diastereomer or other stereoisomeric forms. Each chiral carbon atom may be defined as (R)- or (S)-based on stereochemistry. The compounds of the invention include all possible isomers, and their racemates and optically pure forms. The preparation of the compounds of the present invention can use a racemate, diastereomer or enantiomer as a raw material or as an intermediate. The optically active isomers may be prepared using chiral synthons or chiral reagents, or by conventional techniques, for example, using methods such as crystallization and chiral chromatography.

The term “deuterate” refers to a deuterium-containing compound that is generated by replacement of one or more available hydrogen atoms in the compound with a corresponding number of deuterium atoms. For example, the—CH₃ on the guanine (G) of the present compound could be deuterated as—CD₃, i.e.,

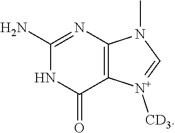 [SMILES: [2H]C([2H])([2H])[n+]1cn(C)c2nc(N)[nH]c(=O)c21]

The compounds of the present invention may be deuterated in a manner known to those of ordinary skill in the art, including in the methods of the present invention described below.

The term “mRNA” means “messenger RNA” and relates to a “transcript” that can be produced by using a DNA template and can encode a peptide or protein. The mRNA is single-stranded, but may contain self-complementary sequences that allow a portion of the mRNA to fold and pair with itself to form a double helix. Generally, mRNA contains 5′-UTR, peptide/protein coding region and 3′-UTR. In the context of the present invention, mRNA is preferably produced from a DNA template by in vitro transcription (IVT). In vitro transcription methods are known to the skilled person, and a variety of in vitro transcription kits are commercially available.

The term “Interleukin-12 (IL-12)” refers to a pluripotent cytokine whose action creates an interconnection between innate and adaptive immunity. IL-12 acts primarily as a 70 kDa heterodimeric protein consisting of two disulfide-linked p35 and p40 subunits. The precursor form of the IL12p40 subunit (also known as IL12B) is 328 amino acids long, while its mature form is 306 amino acids long. The precursor form of the IL12p35 subunit (also known as IL12A) is 219 amino acids long, while the mature form is 197 amino acids long. The genes for the IL12p35 and p40 subunits are located on different chromosomes and are regulated independently of each other. IL-12 consists of a bundle of four alpha helices. It is a heterodimeric cytokine encoded by two independent genes, IL12A (p35) and IL12B (p40). Active heterodimers (termed ‘p70’) and homodimers of p40 are formed after protein synthesis.

The term “coding sequence” refers to a nucleic acid (e.g., mRNA or DNA molecule) coding sequence that encodes a polypeptide. The coding sequence may further include initiation and termination signals operably linked to regulatory elements including a promoter and polyadenylation signals capable of directing expression in cells of the individual or mammal to which the nucleic acid is administered. The coding sequence may also include a sequence encoding a signal peptide.

The term “linker” (including the linker coding sequence linking IL12B polypeptide and IL12A polypeptide mentioned herein) refers to a group of atoms, for example, 10-1,000 atoms, and can be composed of atoms or groups such as, but not limited to, carbon, amino, alkylamino, oxygen, sulfur, sulfoxide, sulfonyl, carbonyl and imine. A linker can be attached at a first end to a nucleobase or a modified nucleoside or nucleotide on a sugar moiety, and at a second end to a payload (e.g., a detectable or therapeutic agent). Linkers can be of sufficient length so as not to interfere with incorporation into the nucleic acid sequence. Linkers can be used for any useful purpose, such as forming polynucleotide polymers (e.g., by linking two or more chimeric polynucleotide molecules or IVT polynucleotides) or polynucleotide conjugates.

The term “untranslated region (UTR)” refers to the untranslated nucleic acid segment of a polynucleotide preceding the start codon (5′UTR) and following the stop codon (3′UTR). In certain embodiments, polynucleotides (e.g., ribonucleic acid (RNA), e.g., messenger RNA (mRNA)) of the present disclosure comprising an open reading frame (ORF) encoding an IL12B polypeptide, an IL12A polypeptide, and/or an IL12A and IL12B fusion polypeptide) also includes UTRs (e.g., 5′ UTRs or functional fragments thereof, 3′ UTRs or functional fragments thereof, or combinations thereof).

The term “transcription” relates to the process in which the genetic code in a DNA sequence is transcribed into RNA. Subsequently, RNA can be translated into protein. According to the present invention, the term “transcription” includes “in vitro transcription”, wherein the term “in vitro transcription” relates to a process in which RNA, especially mRNA, is synthesized in vitro in a cell-free system (preferably using a suitable cell extract). According to the present invention, the RNA used in the present invention can be obtained by in vitro transcription of a suitable DNA template. The promoter used to control transcription can be any promoter of any RNA polymerase. Some specific examples of RNA polymerases are T7, T3 and SP6 RNA polymerases. Preferably, in vitro transcription according to the present invention is controlled by a T7 or SP6 promoter. The DNA template for in vitro transcription can be obtained by cloning nucleic acid, and introducing it into a suitable vector for in vitro transcription.

The term “promoter” refers to a nucleic acid sequence located upstream or 5′ to a translational start codon of an open reading frame (or protein-coding region) of a gene and that is involved in recognition and binding of RNA polymerase II and other proteins (trans-acting transcription factors) to initiate transcription. The promoter refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one skilled in the art to act directly or indirectly to regulate the amount of transcription from the promoter.

The term “excipient” refers to all substances in a pharmaceutical composition which are not active agents (e.g., which are therapeutically inactive ingredients that do not exhibit any therapeutic effect in the amount/concentration used), such as, e.g., salts, carriers, binders, lubricants, thickeners, surface active agents, dispersing agents, preservatives, emulsifiers, buffering agents, wetting agents, flavoring agents, colorants, stabilizing agents (such as RNase inhibitors) or antioxidants all of which are preferably pharmaceutically acceptable.

The term “lipid nanoparticle (LNP)” refers to a vesicle formed by one or more lipid components. Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids (e.g., MC-3), phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems, thus driving biodistribution and cell uptake properties.

### EXAMPLES

### Method of Synthesis

5′-cap structures synthesis protocol

### Example 1

Synthesis of 7mGppp(locked)Ap(locked)ApG (GL-Cap 1)
Synthesis of p(locked)Ap(locked)ApG

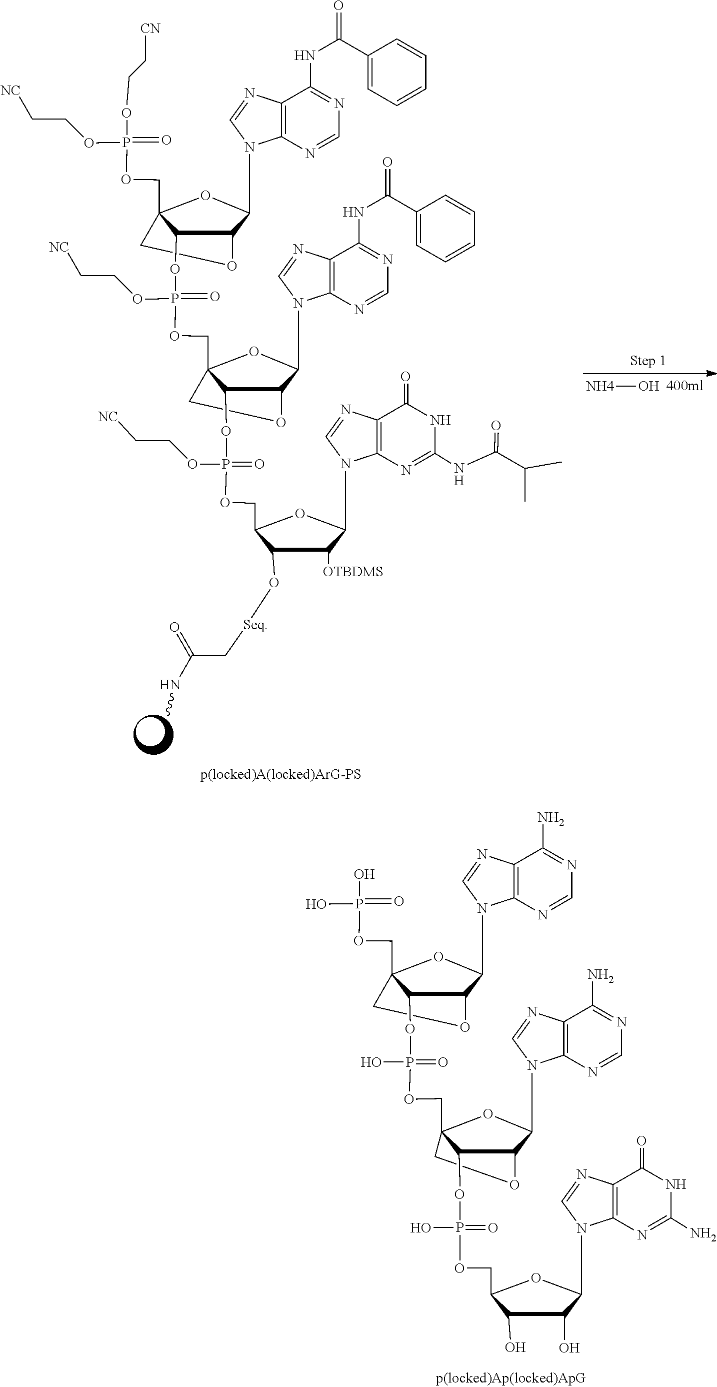

In a 1 L bottle, p(locked)Ap(locked)A G-PS (1.60 g from GENERAL Biosystems (Anhui) and Corporation Limited) and 30% ammonia aqueous solution (350 mL) was added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (405 mg, 388 μmol). MS(m/z): found 1047 [M+H+], 1H NMR (400 MHZ, D2O): δ8.11 (s, 1H), 7.92 (s, 1H), 7.63 (s, 1H), 7.62 (s, 1H), 7.38 (s, 1H), 5.86 (s, 1H), 5.62 (d, J=4.56 Hz, 1H), 5.52 (s, 1H), 5.10 (s, 1H), 4.50 (d, J=5.32 Hz, 1H), 4.31-3.99 (m, 13H), 3.14-3.08(q, 19H), 1.20 (t, 30H). 31P NMR (162 MHz, D2O) δ 0.84(S, 1P), −2.07(S, 1P), −2.15(S, 1P).

Synthesis of 7mGppp(locked)Ap(locked)ApG (GL-Cap 1)

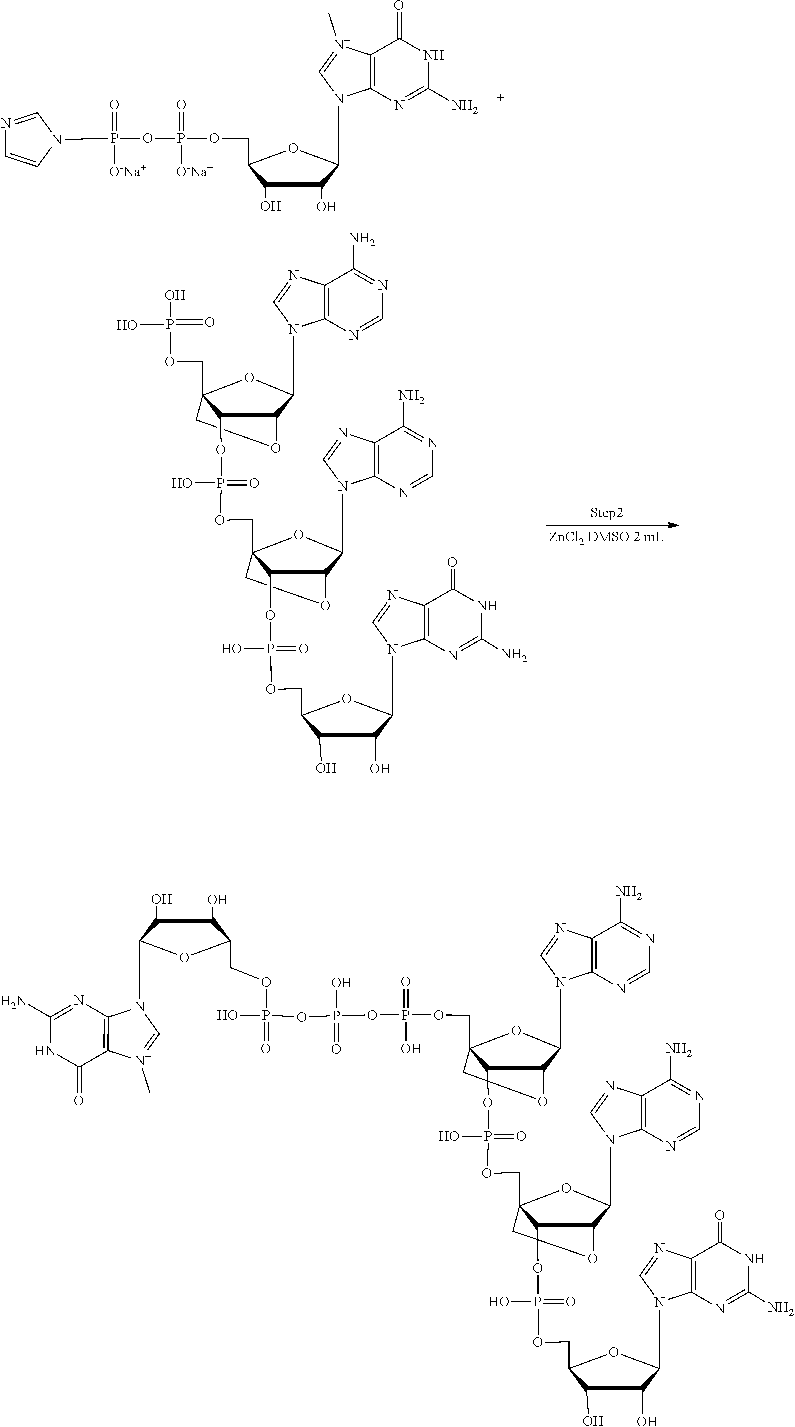

In a 5 mL vial, to a mixture of 7m-GDP-Imidazole (35.5 mg, 67 μmol), p(locked)Ap(locked)ApG TEA (62.7 mg, 60 μmol) and zinc chloride (240 mg, 1.8 mmol) was added dry DMSO (2 mL), and then the whole mixture was stirred at 35° C. for 20 h. The reaction mixture was added to a solution of EDTA disodium (804 mg, 8.0 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to 27 mg white solid. The crude product was purified on HPLC to yield ammonium salt (16 mg, 10.77 μmol). Yield 18.0%. MS(m/z): found 1486 [M+H+]. 1H NMR (400 MHZ, D2O): δ 9.00 (s, 1H), 8.00 (s 1H), 7.96 (s, 1H), 7.64 (s, 1H), 7.62 (s, 1H), 7.38 (s, 1H), 5.62 (s, 1H), 5.61-5.55 (m, 3H), 5.02 (s, 1H), 4.70-3.99 (m, 19H), 3.97 (s, 3H), 31P NMR (162 MHZ, D2O) δ −2.12(s,2P), −11.44(d, 2P), −22.71(t, 1P).

### Example 2

Synthesis of 7mGppp(2′-F)Ap(2′-F)ApG (GL-Cap 2)
Synthesis of p(2′-F)Ap(2′-F)ApG

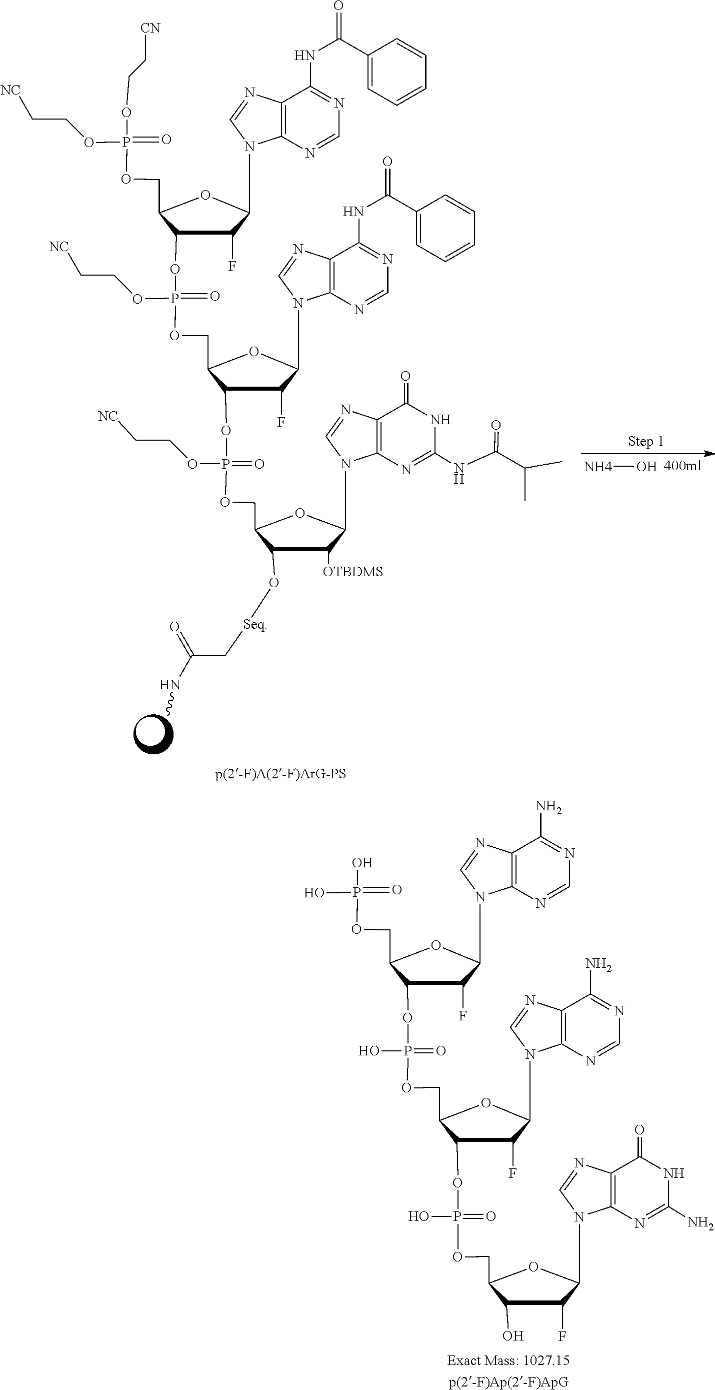

In a 1 L bottle, p(2′F)Ap(2′F)A G-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (253 mg, 247 μmol). MS(m/z): found 1027 [M+H+], 1H NMR (400 MHZ, D2O): δ 8.31 (s, 1H), 7.89 (s, 2H), 7.66 (s, 1H), 7.620 (s, 1H), 6.09 (d, J=14.36 Hz, 1H), 5.99 (d, J=15.8 Hz, 1H), 5.60-5.48 (m, 2H), 5.26-5.13 (m, 1H), 4.61 (m, 14H), 3.14-3.08(q, 22H), 1.20 (t, 32H). 31P NMR (162 MHZ, D2O): δ 1.33(S, 1P), −1.40(s, 1P), −1.76(S,1P).

Synthesis of 7mGppp(2′-F)Ap(2′-F)ApG (GL-Cap 2)

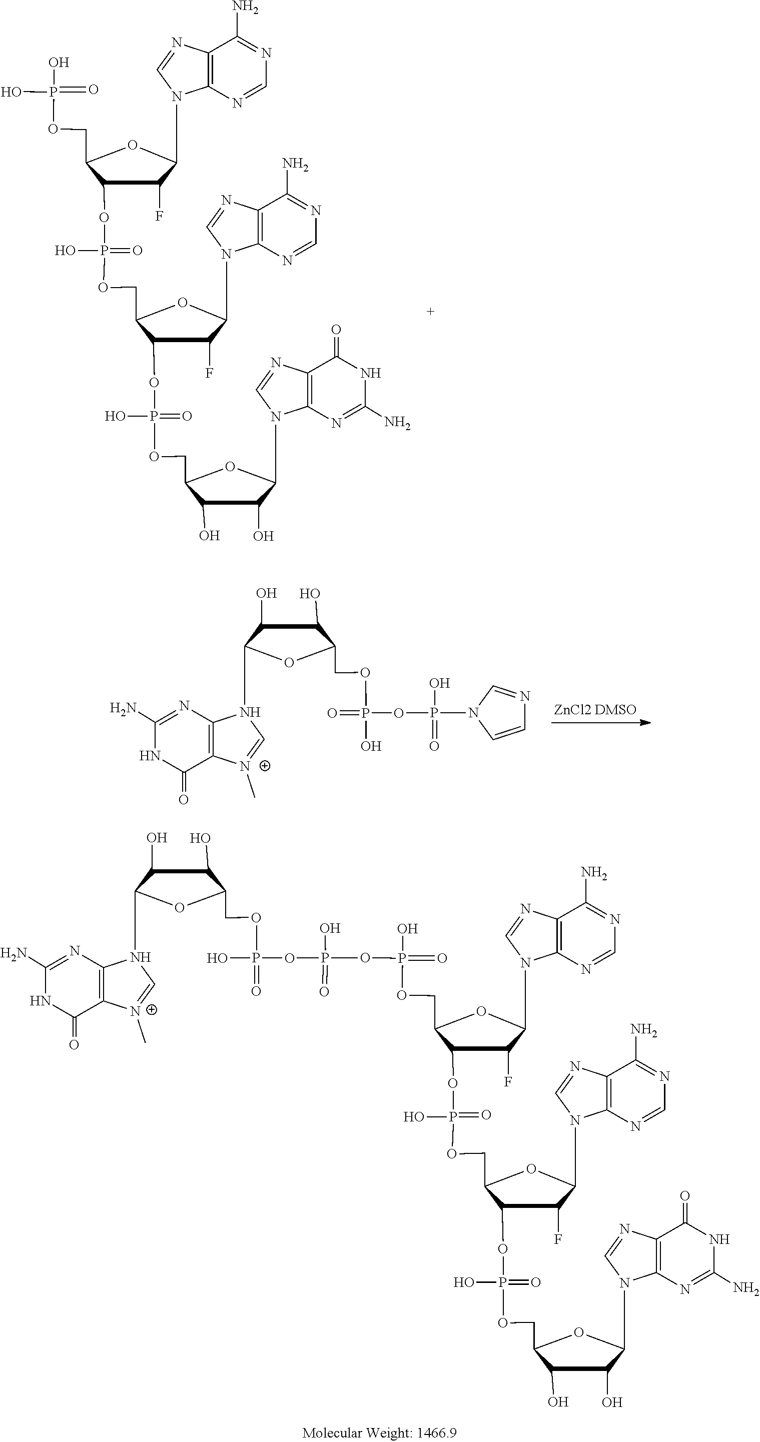

In a 5 mL vial, to a mixture of 7m-GDP-Imidazole (29.5 mg, 55 μmol), p(2′-F)Ap(2′-F)ApG TEA (50.6 mg, 49.5 μmol) and zinc chloride (200 mg, 1.5 mmol) was added dry DMSO (2 mL), and then the whole mixture was stirred at 35° C. for 20 h. The reaction mixture was added to a solution of EDTA disodium (670 mg, 1.8 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to 26 mg white solid. The crude product was purified on HPLC to yield ammonium salt. (9.3 mg, 6.3 μmol). Yield 12.80%. MS (m/z): found 1468 [M+H+], 1H NMR (400 MHZ, D₂O): δ 9.01 (s, 0.59H), 8.27 (s, 1H), 8.02 (s, 1H), 7.99 (s, 1H), 7.80 (s, 1H), 7.69 (s, 1H), 6.04 (dd, J=15.6, 2H), 5.83 (d, J=3.64, 1H), 5.60 (d, J=5.88, 1H), 5.46-5.29 (m, 2H), 4.70-3.97 (m, 22H), 31P NMR (162 MHz, D2O) δ−1.38 (s, 1P), −2.13 (s, 1P), −11.26 (d, 2P), −22.58 (t, 1P).

### Example 3

Synthesis of 7mGppp(2′-F)Ap(locked)ApG (GL-Cap 3)
Synthesis of p(2′-F)Ap(locked)ApG

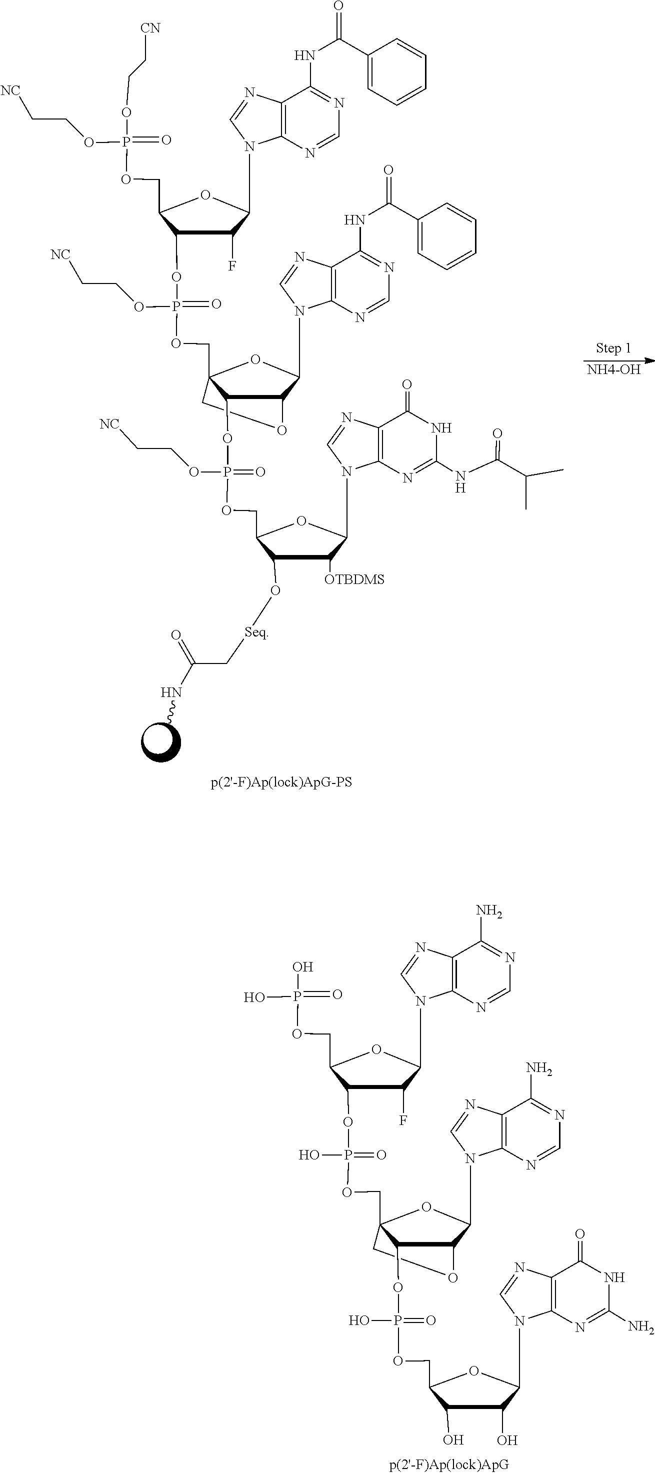

In a 1 L bottle, p(2′F)Ap(locked)ApG-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (236 mg, 228 μmol). MS (m/z): found 1037 [M+H+], 1H NMR (400 MHZ, D2O): δ8.28 (s, 1H), 7.88 (s, 1H), 7.64 (s, 1H), 7.57 (s, 1H), 6.17 (d,13.8 Hz, 1H), 5.79 (s, 1H), 5.66 (s, 1H), 5.62 (d, J=4.52 Hz, 1H), 4.61 (d, J=5.16 Hz, 1H), 4.48 (d, J=6.52 Hz, 1H), 4.32-4.01 (m, 11H), 3.14-3.08 (q, 17H), 1.20 (t, 26H). 31P NMR (162 MHZ, D2O): δ 0.17 (s, 1P), −1.30 (S, 1P), −2.09 (s, 1P).

Synthesis of 7mGppp(2′-F)Ap(locked)ApG (GL-Cap 3)

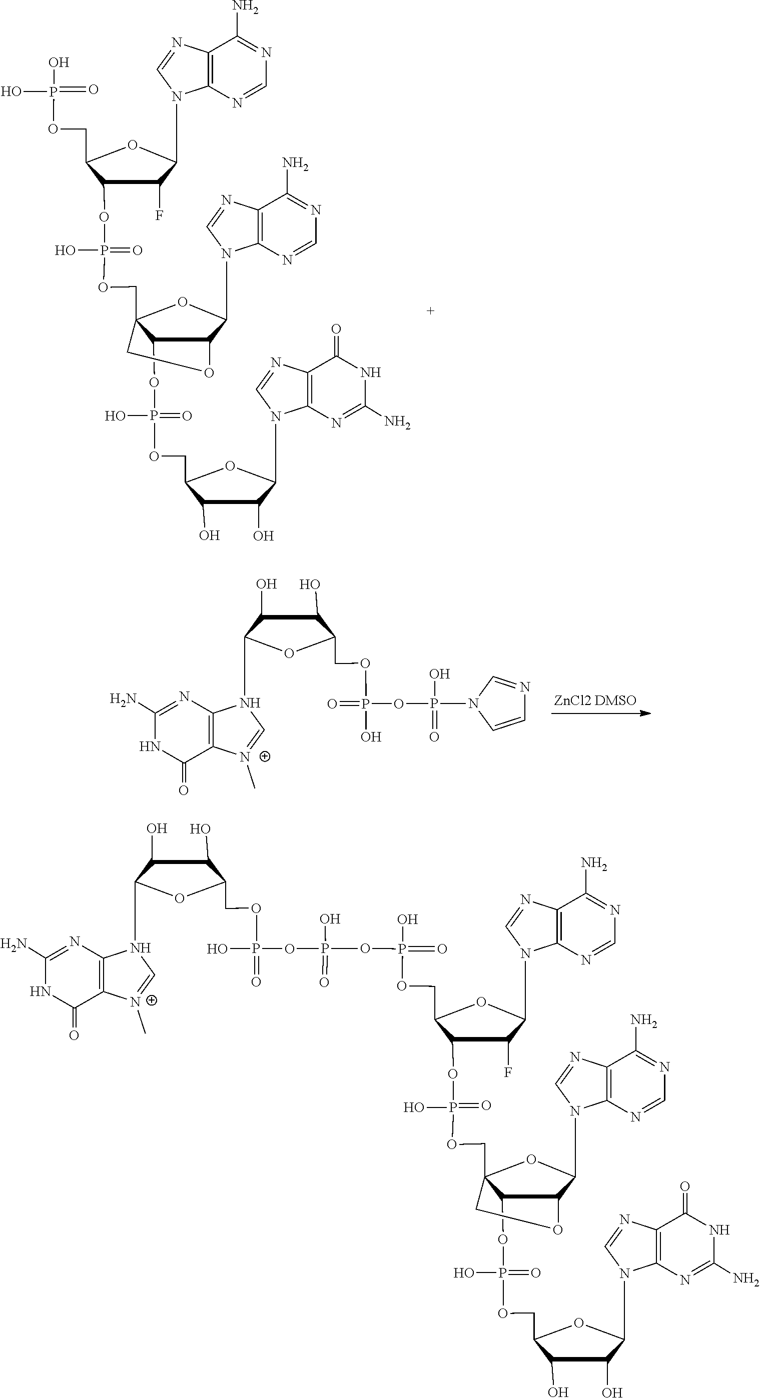

In a 5 mL vial, to a mixture of 7m-GDP-Imidazole (48.3 mg, 87.5 μmol), p(2′-F)Ap(locked)ApG TEA (72.5 mg, 70 μmol) and zinc chloride (235 mg, 1.75 mmol) was added dry DMSO (2 mL), and then the whole mixture was stirred at 35° C. for 20 h. The reaction mixture was added to a solution of EDTA disodium (782 mg, 2.1 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to 24 mg white solid. The crude product was purified on HPLC to yield ammonium salt (16 mg, 10.8 μmol). Yield 15.4%. MS (m/z): found 1477 [M+H⁺]. ¹H NMR (400 MHZ, D₂O): δ 8.96 (s, 0.7H), 8.33 (s 1H), 8.10 (s, 1H), 7.79 (s, 1H), 7.69 (s, 1H), 7.65 (s, 1H), 6.14 (d, J=13.32 Hz, 1H), 5.74-5.68 (m, 3H), 5.63 (d, J=5.68 Hz, 2H), 4.70 (s, 2H), 4.58 (d, J=5.48 Hz, 1H), 4.50 (d, J=6.12 Hz, 1H), 4.43-4.05 (m, 18H), 3.90 (s, 3H), ³¹P NMR (162 MHZ, D₂O) δ−1.28 (S, 1P), −1.97 (S, 1P), −11.38 (d, 1P), −22.61 (t, 1P).

### Example 4

Synthesis of 7mGppp(locked)Ap(2′-F)ApG (GL-Cap 4)
Synthesis of p(locked)Ap(2′-F)ApG

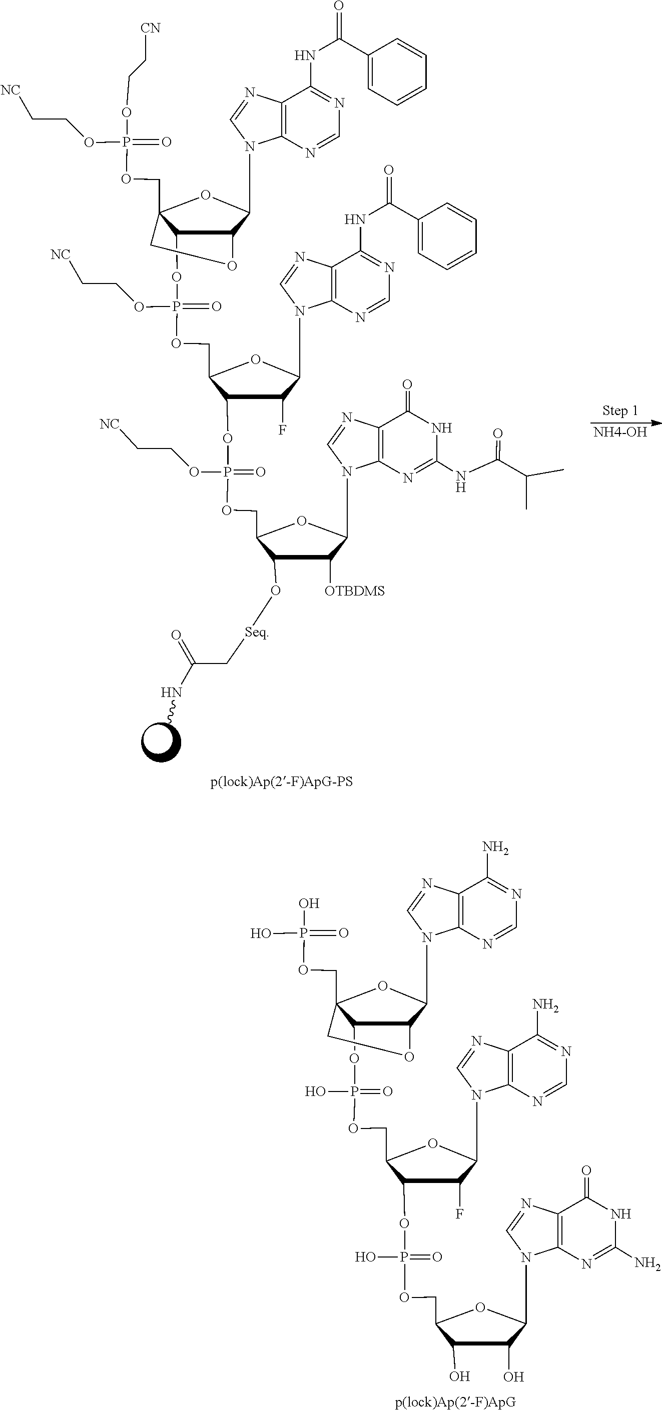

In a 1 L bottle, p(locked)Ap(2′F)ApG-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (265 mg, 256 μmol). MS (m/z): found 1037 [M+H+], 1H NMR (400 MHZ, D₂O): δ8.13 (s, 1H), 7.91 (s, 1H), 7.85 (s, 1H), 7.59 (s, 1H), 7.58 (s, 1H), 5.95 (d, 14.96 Hz, 1H), 5.84 (s, 1H), 5.55 (d, J=6.32 Hz, 1H), 5.20-5.07 (m, 1H), 5.06 (d, J=5.76 Hz, 1H), 4.53 (t, 5.56 Hz, 1H),4.44-3.99 (m, 11H), 3.14-3.08 (q, 17H), 1.20 (t, 26H). ³¹P NMR (162 MHZ, D₂O): δ 0.23 (S, 1P), −1.44 (S, 1P), −2.55 (S, 1P)

Synthesis of 7mGppp(locked)Ap(2′-F)ApG (GL-Cap 4)

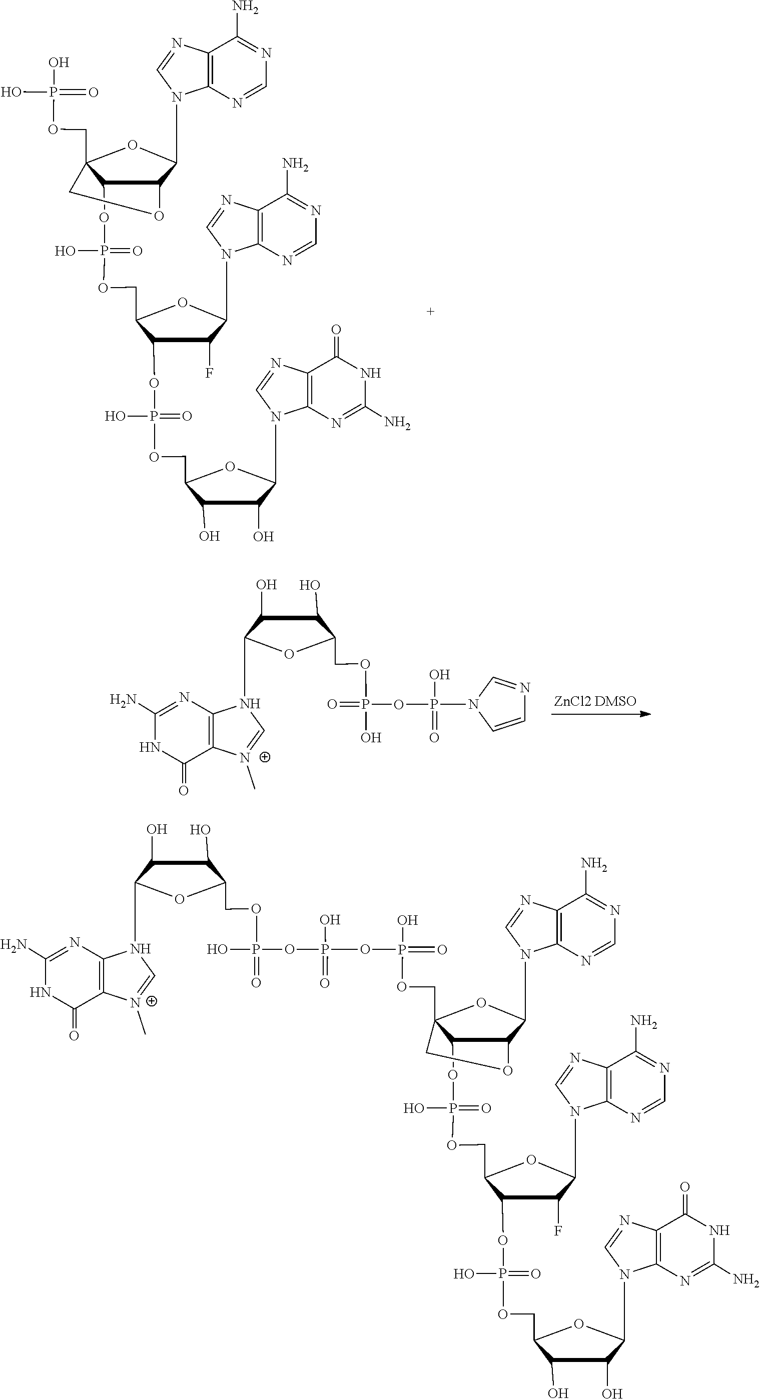

In a 5 mL vial, to a mixture of 7m-GDP-Imidazole (45.5 mg, 82.5 μmol), p(locked)Ap(2′-F)ApG TEA (68.3 mg, 66 μmol) and zinc chloride (221 mg, 1.65 mmol) was added dry DMSO (2 mL), and then the whole mixture was stirred at 35° C. for 20 h. The reaction mixture was added to a solution of EDTA disodium (737 mg, 2.0 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to 34 mg white solid. The crude product was purified on HPLC to yield ammonium salt (23 mg, 15.6 μmol). Yield 23%. MS (m/z): found 1477 [M+H+], 1H NMR (400 MHZ, D2O): δ 9.03 (s, 0.58H), 7.99 (s 1H), 7.90 (s, 1H), 7.81 (s, 1H), 7.56 (s, 1H), 7.55 (s, 1H), 5.93 (d, J=15.04 Hz 1H), 5.81 (d, J=4.28 Hz 1H), 5.55 (d, J=6.32 Hz 1H), 5.52 (s, 1H), 5.17-5.04 (m, 1H), 4.92 (s, 1H), 4.70-4.01 (m, 23H), 31P NMR (162 MHZ, D2O) δ −1.45 (s, 1P), −2.78 (S, 1P), −11.50 (d, 2P), −22.84 (t, 1P).

### Example 5

Synthesis of 7mGppp(locked)ApG (GL-Cap 5)
Synthesis of p(locked)ApG

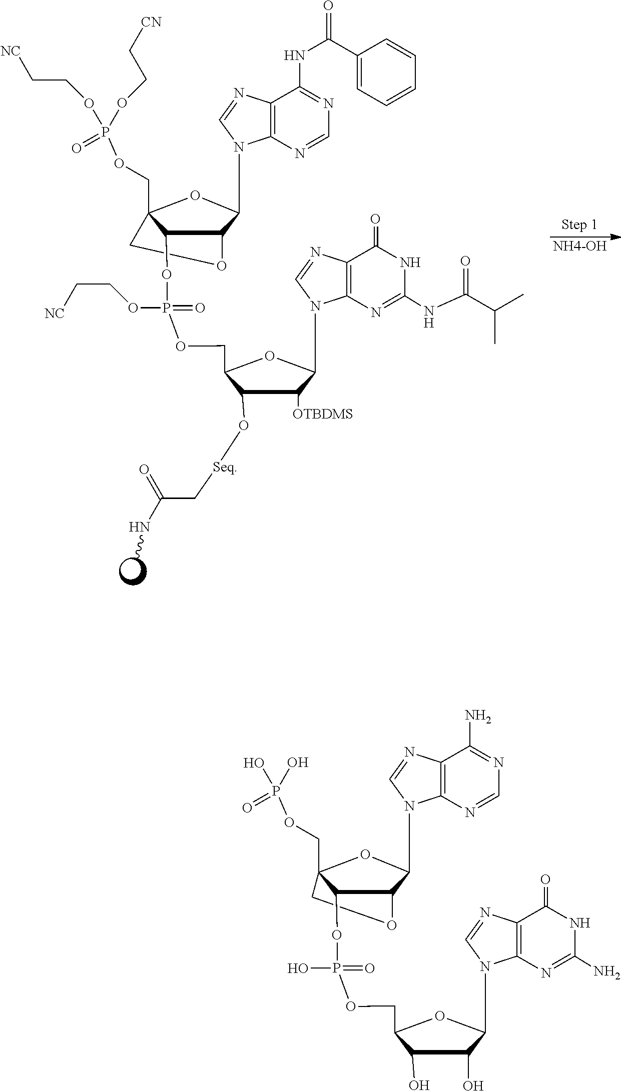

In a 1 L bottle, p(locked)ApG-PS (2.0 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid TEA salt (193 mg, 274 μmol).

MS (m/z): found 705 [M+H+], 1H NMR (400 MHZ, D2O): δ8.15 (s, 1H), 7.92 (s, 1H), 7.71 (s, 1H), 5.92 (s, 1H), 5.63 (d, J=4.4 Hz, 1H), 4.95 (s, 1H), 4.33-4.05 (m, 9H), 3.14-3.00 (q, 16H), 1.20 (t, 24H). 31P NMR (162 MHZ, D2O) δ 1.93 (s, 1P), 2.29 (S, 1P).

Synthesis of 7mGppp(locked)ApG (GL-Cap 5)

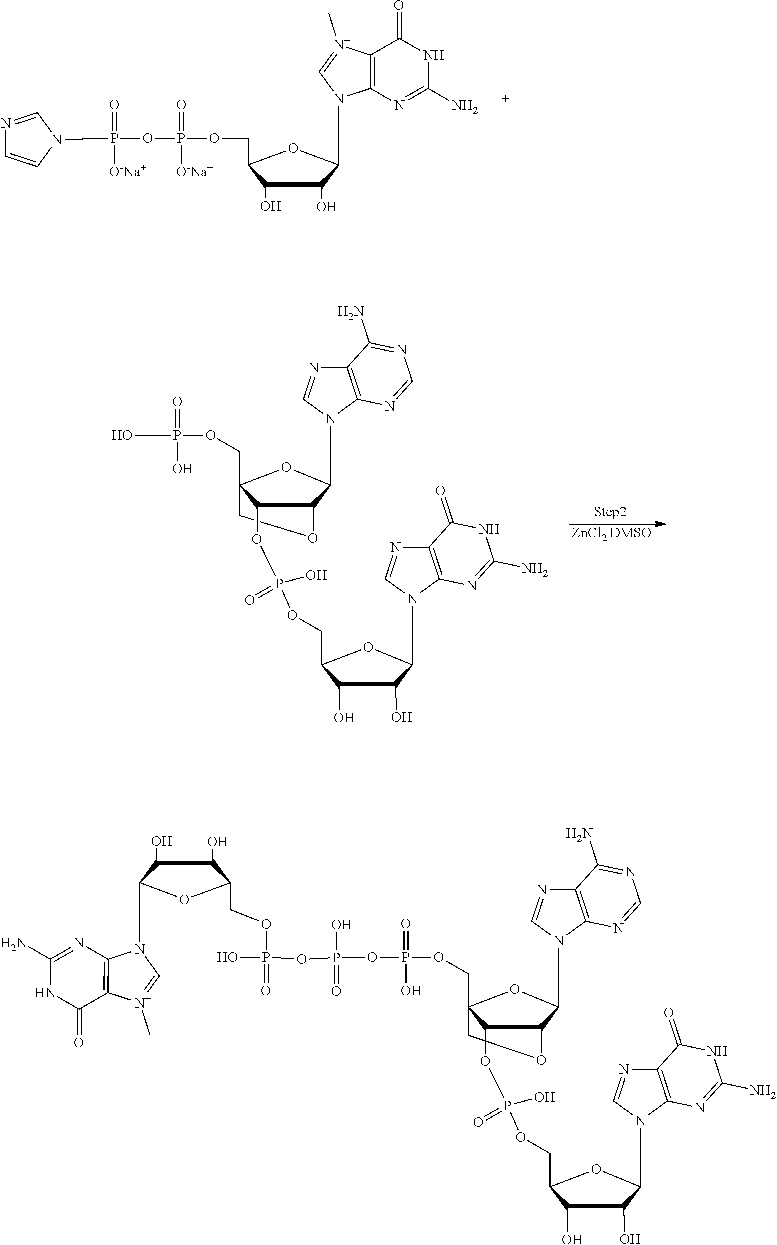

In a 5 mL vial, to a mixture of 7m-GDP-Imidazole (140 mg, 228 μmol), p(locked)ApG TEA (160 mg, 227 μmol) and zinc chloride (915 mg, 6.84 mmol) was added dry DMSO (2 mL), and then the whole mixture was stirred at 35° C. for 20 h. The reaction mixture was added to a solution of EDTA disodium (3.0 g, 8.0 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to 106 mg white solid. The crude product was purified on HPLC to yield ammonium salt (48.3 mg, 42 μmol). Yield 18.5%. MS (m/z): found 1144 [M+H+]. 1H NMR (400 MHZ, D2O): δ9.05 (s, 1H), 8.11 (s, 1H), 8.01 (s, 1H), 7.76 (s, 1H), 5.80 (d, J=4.1 Hz, 1H), 5.67 (s, 1H), 5.62 (d, J=4.4 Hz 1H), 4.85 (s, 1H), 4.70 (m, 2H), 4.51-4.02 (m, 13H), 4.00 (s, 3H). 31P NMR (162 MHZ, D2O) δ −1.96 (S, 1P), −11.53 (d, 2P), −22.95 (t, 1P).

### Example 6

Synthesis of 7m-3′-MOEGppp(locked)ApG (GL-Cap 6)

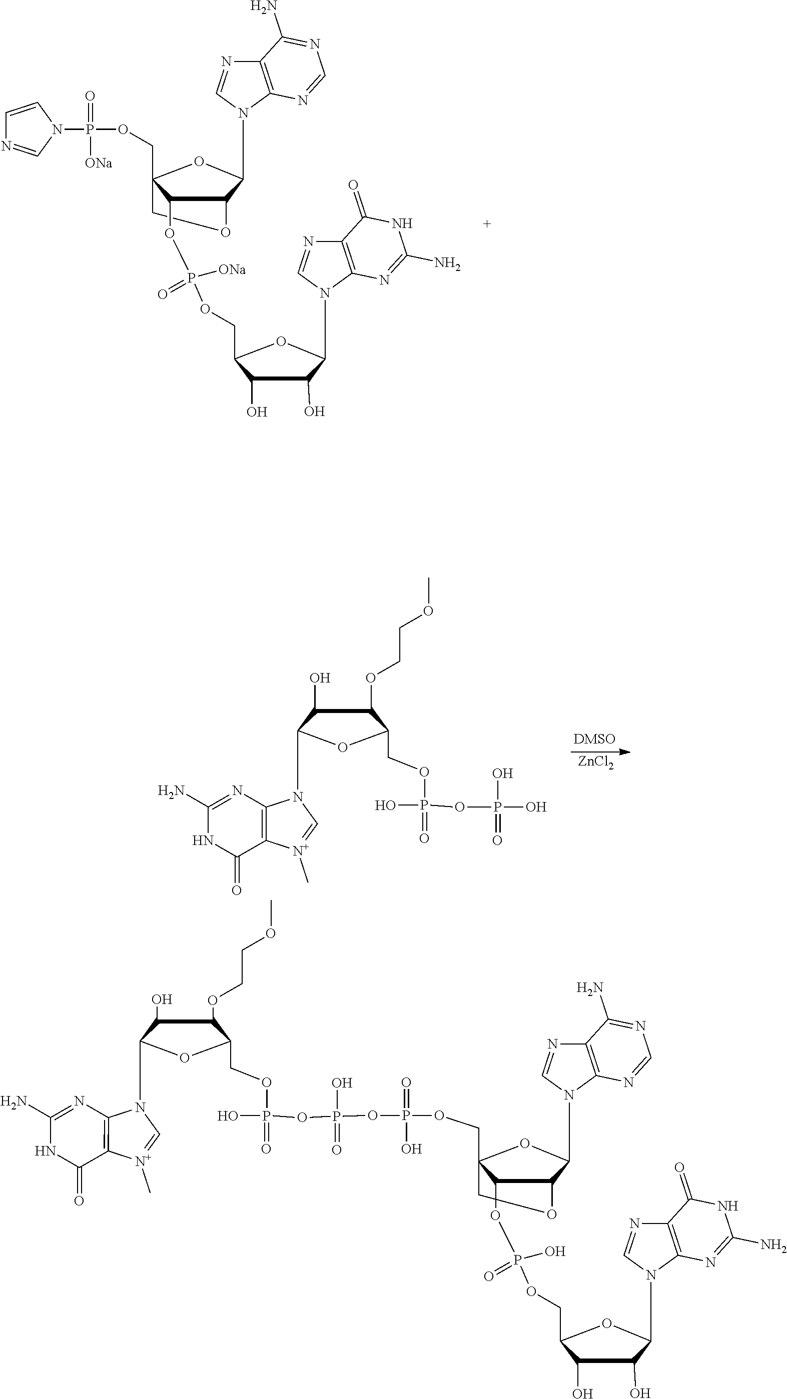

In a 5 mL vial, to a mixture of 7m-2′-MOE-GDP TEA (20.7 mg, 40.1 μmol), p(locked)ApG-Imidazole (32.0 mg, 40.1 μmol) and zinc chloride (164 mg, 1.2 mmol) was added dry DMSO (0.8 mL), and then the whole mixture was stirred at 35° C. for 20 h. The reaction mixture was added to a solution of EDTA disodium (580 mg, 1.6 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to crude product. The crude product was purified on HPLC to yield ammonium salt (7.5 mg, 6 μmol). Yield 15%. MS (m/z): found 1203 [M+H+], 1H NMR (400 MHZ, D2O): δ 8.99 (s, 1H), 8.37 (s 1H), 7.90 (s, 1H), 7.87 (s, 1H), 7.66 (s, 1H), 5.78 (d, 1H), 5.60 (m, 2H), 4.80 (s, 1H), 4.54-4.14 (m, 17H), 4.01 (S, 3H), 3.7 (m, 2H), 3.57 (m, 2H), 3.32 (s, 3H), 31P NMR (162 MHz, D2O) δ −2.02 (s, 1P), −11.57 (d, 2P), −22.77 (m, 1P)

### Example 7

Synthesis of 7m-2′F-Gppp(locked)ApG (GL-Cap 7)

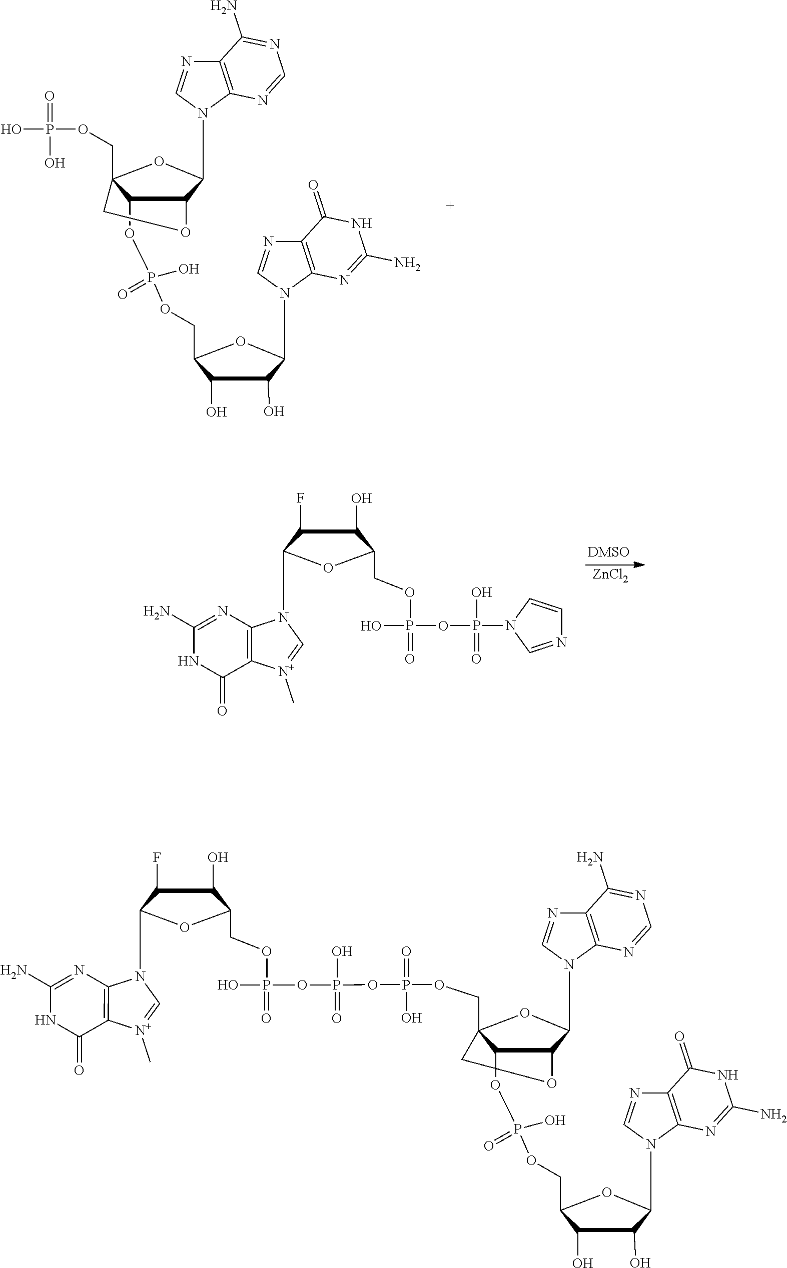

In a 5 mL vial, to a mixture of 7m-2′-F-GDP-Imidazole (35 mg, 66 μmol), p(locked)ApG (60.0 mg, 85 μmol) and zinc chloride (228 mg, 1.7 mmol) was added dry DMSO (1.0 mL), and then the whole mixture was stirred at 35° C. for 20 h. The reaction mixture was added to a solution of EDTA disodium (830 mg, 2.1 mmol) in 100.0 mL of water at 0° C. The resulting aqueous solution was adjusted to pH 6.0 and loaded on a DEAE Sephadex column. The desired product was eluted using a linear gradient of 0-1M TEAB and the fractions containing the product were pooled, evaporated and concentrated to crude product. The crude product was purified on HPLC to yield ammonium salt (5 μmol). Yield 6%. MS (m/z): found 1147 [M+H+], 1H NMR (400 MHZ, D2O): 8.03 (s 1H), 7.89 (s, 1H), 7.68 (s, 1H), 6.09 (d, 1H), 5.68 (s, 1H), 5.61 (d, 1H), 5.32-5.19 (m, 1H), 4.85 (s, 1H), 4.57-4.00 (m, 17H), 31P NMR (162 MHZ, D2O) δ −2.04 (s, 1P), −11.51 (D,2P), −22.73 (t, 1P)

### Example 8

Synthesis of 7m-2-′MOE-Gppp(locked)ApG (GL-Cap 8)

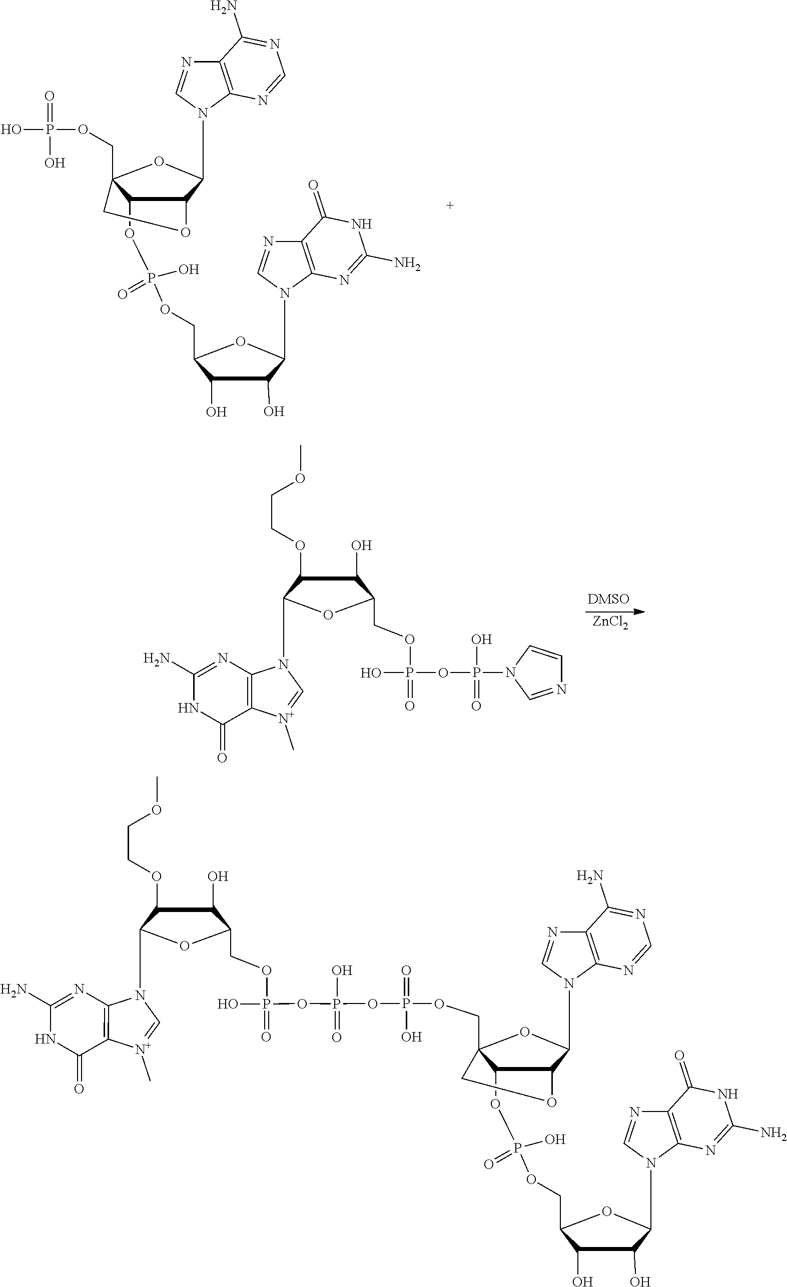

In a 5 mL vial, to a mixture of 7m-2′-MOE-GDP-Imidazole (43 mg, 76 μmol), p(locked)ApG (55 mg, 76 μmol) and zinc chloride (215 mg, 1.5 mmol) was added dry DMSO (1 mL), and then the whole mixture was stirred at 35° C. for 40 h. The reaction mixture was added to a solution of EDTA disodium (860 mg, 2.2 mmol) in 100.0 mL of water at 0° C. The resulting aqueous solution was adjusted to pH 6.0 and loaded on a

DEAE Sephadex column. The desired product was eluted using a linear gradient of 0-1M TEAB and the fractions containing the product were pooled, evaporated and concentrated to crude product. The crude product was purified on HPLC to yield ammonium salt (4 μmol). Yield 5%. MS (m/z): found 1203 [M+H+], 1H NMR (400 MHZ, D2O): 8 9.02 (S, 1H), 8.16 (s, 1H), 8.04 (s 1H), 7.80 (s, 1H), 5.89 (d, 1H), 5.76 (s, 1H), 5.65 (d, 1H), 4.86 (s, 1H), 4.70-4.01 (m, 12H), 4.61-4.57 (m, 2H), 4.41-3.96 (m, 14H), 31P NMR (162 MHz, D2O) δ −1.32 (S, 1P), −11.50 (d, 2P), −22.74 (t, 1P).

### Example 9

Synthesis of 7m-3′-MOE-5′-RmGppp(2′-Ome)ApG (GL-Cap 9)

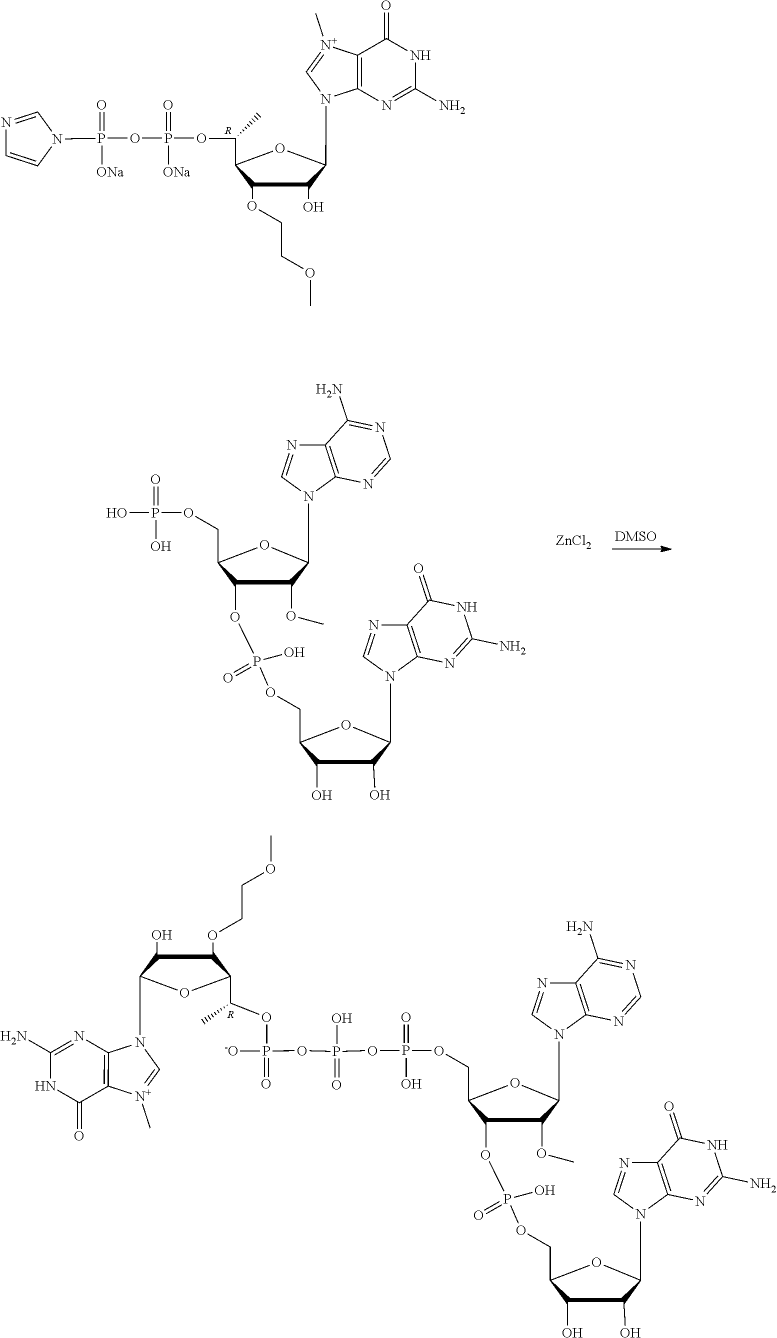

In a 4 mL vial, to a mixture of 7m-3′-MOE-5′-RmGDP-Im (25 μmol), pAmG-TEA (20 μmol) and zinc chloride (67 mg, 500 μmol) was added dry DMSO (500 μL) and the whole mixture was stirred at 35° C. for 24 h. HPLC detected ˜40% conversion so the mixture was stirred for an additional 24 h. The whole mixture was diluted with water (50 mL), treated with bound EDTA quadrapure resin (0.5 g), and concentrated and purified by prep-HPLC. Fractions in the major peak were combined and freeze dried twice to yield 4.0 μmol product, as ammonium salt. MS(m/z): found 1219 [M+H+], 1H NMR (400 MHz, D₂O): δ 9.27 (S, 1H), 8.41 (s 1H), 8.19 (s, 1H), 7.96 (s, 1H), 6.05 (d, 1H), 5.92 (d, 1H), 5.85 (d, 1H), 4.70-3.97 (m, 14H), 4.08 (S, 3H), 3.44 (S, 3H), 3.30 (S, 3H), 1.29 (d, 3H), ³¹P NMR (162 MHZ, D₂O) δ −1.12 (S, 1P), −11.30 (d, 2P), −21.31 (t, 1P).

### Example Activity Assessment

### Experimental Example 1

IL-12-GL-Cap 5 and IL-12-GL-Cap 9 mRNAs in vitro transcription (IVT)

mRNAs were synthesized via co-transcriptional in-vitro transcription reaction by mixing in all components including buffer, NTPs (ATP, GTP, UTP, CTP or modified nucleotides), cap (GL-Cap 5 or GL-Cap 9), linearized DNA template, T7 RNA polymerase, RNase inhibitor, and phosphatase at 37° C. with agitation at 300 rpm for 2 hours. After the reaction was done, DNase I was added to the reaction and incubated at 37° C. for 30 minutes. Then the reaction mixture was diluted with an appropriate amount of loading buffer and heated at 65° C. for 15 minutes before loading onto the Oligo dT column on the Acta system. After binding and washing steps on the Oligo dT column, the mRNA samples were eluted with water. The eluted mRNA concentration was normalized to 1 mg/mL and ready for formulation.

### Experimental Example 2

IL-12-GL-Cap 5 and IL-12-GL-Cap 9 mRNAs were formulated into LNPs

IL-12-GL-Cap 5 and IL-12-GL-Cap 9 mRNAs were formulated into LNPs using the NanoAssemblr microfluidic system (Precision NanoSystems, Vancouver, BC, Canada). In brief, the lipids MC-3, DSPC, Cholesterol, and DMG-PEG2000 were dissolved in 100% ethanol and were mixed at a molar ratio of 50% MC-3, 38.5% Cholesterol, 10% DSPC, and 1.5% DMG-PEG2000 with a final MC-3 concentration of 12.81 mg/mL. The IL-12 mRNAs were prepared in 20 mM acidic sodium malic acid buffer containing mRNA (0.451 mg/mL, pH 3.0) respectively. The aqueous and ethanol solutions were mixed in a 3:1 volume ratio at a mixing rate of 12 mL/min. Formulations were further dialyzed against PBS (10 mM, pH 7.4) in dialysis cassettes overnight at 4° C. The particle size, PDI, and Zeta potential of formulations were determined using dynamic light scattering (DLS) using a zeta-sizer (Malvern Panalytical Ltd., Cambridge, United Kingdom). The concentration of mRNA and encapsulation efficiency were characterized by Ribogreen assay.

### Experimental Example 3

In vitro mRNA-derived IL-12p70 expression and bioactivity

The secreted human IL-12p70 protein is measured at 24 hours in the supernatant of HeLa cells transfected with 500 ng hIL-12 mRNA per well on a 24-well plate, using Lipofectamine Messenger Max (Invitrogen) or LNP. In vitro h(m)IL12p70 expression is quantified by DuoSet ELISA (R&D Systems).

Hela cells were transfected with 0.5 μg of MC3-based LNP-formulated hIL-12 mRNA (hGL-001) (wherein the sequence encoding IL-12 is SEQ ID NO:8) co-transcriptionally capped with GL-Cap 9, or hIL-12 mRNA (hMD-001) co-transcriptionally capped with CleanCap®. The IL-12 protein expression in the cell supernatants at 24 hours post-transfection was quantified by ELISA.

Hela cells were transfected with 0.5 μg of Messenger Max. hGL-001 mRNA was co-transcriptionally capped with either GL-Cap 9 or GL-Cap 5, or hMD-001 mRNA was co-transcriptionally capped with CleanCap®. The IL-12 protein expression in the cell supernatants at 48 hours post-transfection was quantified by ELISA.

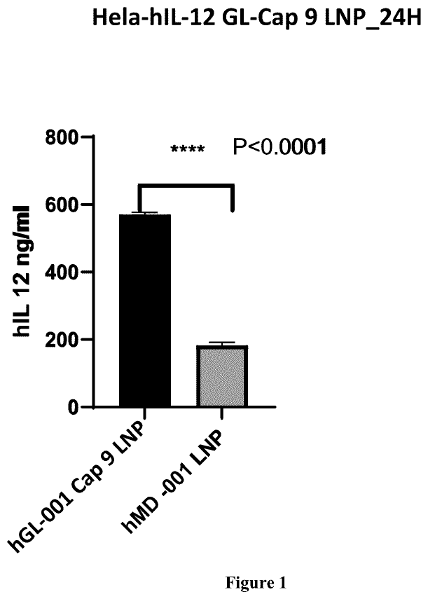

As shown in FIG. 1, the GL-Cap 9-capped hGL-001 delivered over 3-fold higher expression than CleanCap®-hMD-001.

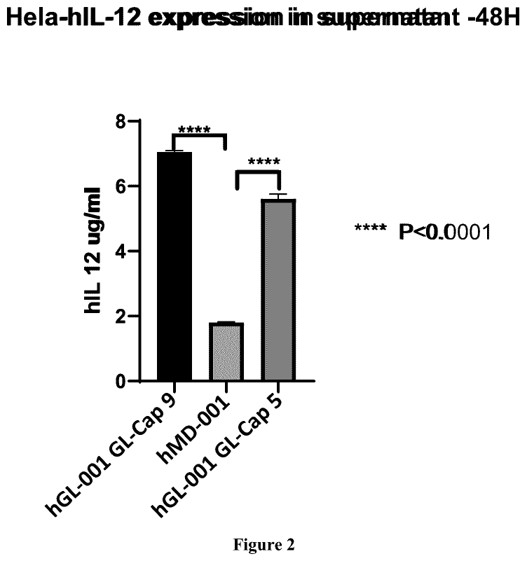

As shown in FIG. 2, the GL-Cap 9-capped hGL-001 delivered over 3-fold higher expression than CleanCap®-hMD-001, and the GL-Cap 5-capped hGL-001 delivered over 2.5-fold higher expression than CleanCap®-hMD-001.

### Experimental Example 4

IL-12-GL-Cap 5 and IL-12-GL-Cap 9 mRNAs LNP Treatment on Syngeneic Tumor Models

All the in vivo experiments were carried out in accordance with the Institutional Animal Care and Use Committee (IACUC) at Biocytogen, Wakefield, MA. For syngeneic models, female C57BL/6 mice (Jackson Laboratory or Charles River UK) or Balb/c mice (Charles River Laboratories) are implanted subcutaneously (SC) with MC38 (colon cancer) tumor cells. Tumor size was periodically measured with calipers, and volumes were calculated using the formula volume=width²×Length/2. IL-12 mRNA was administered intratumorally. Survival events were either recorded based on sudden death or humane endpoints (total tumor volume>2,000 mm³, body weight loss>20%, ulcerations and other clinical signs). Kaplan-Meier survival plots are based on the total number of animals enrolled in each group.

As soon as the MC38 tumors reached 100 mm³, our GL-Cap 5 capped IL-12 mRNA (GL-001) (wherein the sequence encoding IL-12 is SEQ ID NO:1) was intratumorally (IT) injected once at a dose of 1 μg. CleanCap-capped Luciferase mRNA and CleanCap-capped IL-12 mRNA (GL-001) were used at the same dose (single IT injection of 1 μg) for comparison.

As soon as the MC38 tumors reached 100 mm³, our GL-Cap 9 capped IL-12 mRNA (GL-001) (wherein the sequence encoding IL-12 is SEQ ID NO:1) was intratumorally (IT) injected once at a dose of 1 μg. CleanCap-capped Luciferase mRNA and CleanCap-capped IL-12 mRNA (GL-001) were used at the same dose (single IT injection of 1 μg) for comparison.

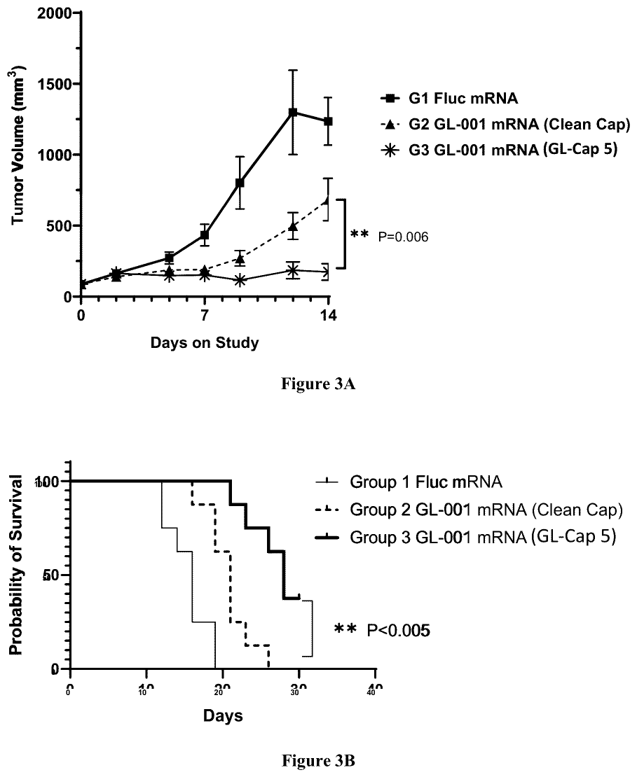

As shown in FIGS. 3A and 3B, GL-Cap 5 capped-GL-001 inhibited tumor growth and improved survival significantly better than CleanCap capped-GL-001 (in FIG. 3A, data is presented as mean±SEM, and in FIG. 3B, data is presented as Kaplan-Meier curves).

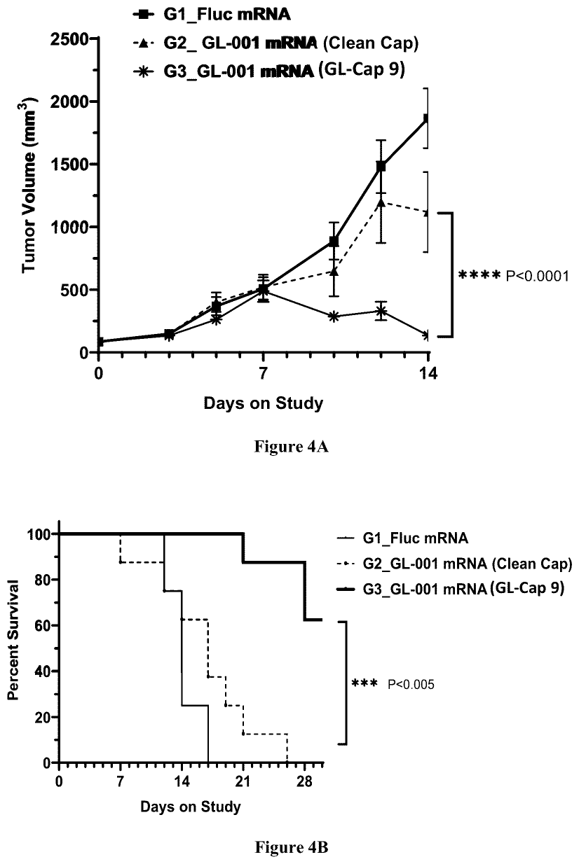

As shown in FIGS. 4A and 4B, GL-Cap 9 capped-GL-001 inhibited tumor growth and improved survival significantly better than CleanCap capped-GL-001 (in FIG. 4A, data is presented as mean±SEM, and in FIG. 4B, data is presented as Kaplan-Meier curves).

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

SEQUENCE LISTING
SEQ ID NO: 1
aggtgtatttactagtaacgtaacagtgttacgtagtgacgtttaa
ctgagactatgggcgtaaaagttctctttgcactcatatgcattg
ctgtggccgaggctatgtgggaacttgaaaaagatgtgtatgtcg
ttgaagtggactggacgcctgacgccccgggagaaacagtgaacc
tcacatgtgacacaccagaggaagatgacatcacgtggacatctg
atcagaggcatggcgtgattggatctgggaagactctaaccataa
ctgttaaggaatttctggacgctgggcagtacacctgccacaaag
gtggtgagaccctgagtcacagccatcttttactccataagaaag
agaatggaatttggtcaactgagatcctgaagaacttcaagaaca
agaccttccttaagtgtgaagcaccaaattattctggtcgcttca
catgcagctggctggtccaaaggaacatggatctgaaatttaaca
tcaaaagcagcagttcatctccagattccagggctgttacctgtg
gcatggcctctctatctgctgagaaagtgacattggaccaaagag
attatgagaagtacagtgtaagctgccaggaggacgtgacttgtc
ccactgcagaggagacactgcctattgagctggcactggaggcga
ggcaacagaacaaatatgaaaactattctactagcttttttattc
gagacatcatcaagccagaccctcccaaaaatctgcaaatgaagc
ctctaaaaaacagtcaggtggaggtgagctgggagtaccccgatt
cttggagcaccccacactcctacttcagtctaaaattcttcgtcc
gtattcagcggaagaaggagaagatgaaagaaactgaagagggct
gcaaccagaagggggcttttctcgtagaaaagacctcaactgaag
tccagtgtaaaggggcaatgtctgtgtgcaggcccaggacagata
ctacaattcatcatgctccaagtgggcatgtgttccctgccgagt
ccgtagtggagggtcagcaggaggatctgccggtggatcacctgg
tggcagtaaacgggtcatccctgtttcggggcctgcccgctgtct
gagccaatccagaaatttgttaaaaaccacagatgacatggtgaa
gacggctcgagaaaagctcaagcactactcctgcacagctgaaga
tattgaccatgaggatataacaagagaccagaccagcacattgaa
gacctgcttgccgttggagctgcacaagaatgaatcttgcctggc
cacccgggagacttcctccactacccgcgggtcctgtcttccccc
ccaaaagacatccctgatgatgaccctctgtctggggagcatcta
tgaagatttaaaaatgtaccagacagagttccaagctatcaatgc
agccctccagaatcacaaccaccagcagatcattctggacaaggg
aatgctggtggccatcgatgagcttatgcagagtctgaaccacaa
tggcgagactcttcggcagaaacccccagtgggggaggcagaccc
atacagggtaaagatgaagctgtgcatcctcttacatgccttttc
tacgcgcgtagtgaccataaacagagtgatgggctatctttcatc
cgcgtgataataggctcgctttcttgctgtccaatttctattaaa
ggttcctttgttccctaagtccaactactaaactgggggatatta
tgaagggccttgagcatctggattctgcctaataaaaaacattta
ttttcattgcaaaaaaaaaaaaaaaaaaaaaaaaaaaaaaaaaaa
aaaaaaaaaaaaaaaaaaaaaaaaaaaaaaaaaaaaaaaaaaaaa
aaaaaaaaaaaaaaaaaaaaaaaaaaaaaaaaaaaaaaaaaaaa
SEQ ID NO: 2
atgtgggaac ttgaaaaaga tgtgtatgtc gttgaagtgg actggacgcc tgacgccccg 60
ggagaaacag tgaacctcac atgtgacaca ccagaggaag atgacatcac gtggacatct 120
gatcagaggc atggcgtgat tggatctggg aagactctaa ccataactgt taaggaattt 180
ctggacgctg ggcagtacac gtgccacaaa ggtggtgaga ccctgagtca cagccatctt 240
ttactccata agaaagagaa tggaatttgg tcaactgaga tcctgaagaa cttcaagaac 300
aagaccttcc ttaagtgtga agcaccaaat tattctggtc gcttcacatg cagctggctg 360
gtccaaagga acatggatct gaaatttaac atcaaaagca gcagttcatc tccagattcc 420
agggctgtta cctgtggcat ggcccctcta tctgctgaga aagtgacatt ggaccaaaga 480
gattatgaga agtacagtgt aagctgccag gaggacgtga cttgtcccac tgcagaggag 540
acactgccta ttgagctggc actggaggcg aggcaacaga acaaatatga aaactattct 600
actagctttt ttattcgaga catcatcaag ccagaccctc ccaaaaatct gcaaatgaag 660
cctctaaaaa acagtcaggt ggaggtgagc tgggagtacc ccgattcttg gagcacccca 720
cactcctact tcagtctaaa attcttcgtc cgtattcagc ggaagaagga gaagatgaaa 780
gtccagtgta aaggcggcaa tgtctgtgtg caggcccagg acagatacta caattcatca 900
tgctccaagt gggcatgtgt tccctgccga gtccgtagt 939
SEQ ID NO: 3
cgggtcatcc ctgtttcggg gcctgcccgc tgtctgagcc aatccagaaa tttgttaaaa 60
accacagatg acatggtgaa gacggctcga gaaaagctca agcactactc ctgcacagct 120
gaagatattg accatgagga tataacaaga gaccagacca gcacattgaa gacctgcttg 180
ccgttggagc tgcacaagaa tgaatcttgc ctggccaccc gggagacttc ctccactacc 250
cgcgggtcct gtcttccccc ccaaaagaca tccctgatga tgaccctctg tctggggagc 300
atctatgaag atttaaaaat gtaccagaca gagttccaag ctatcaatgc agccctccag 360
aatcacaacc accagcagat cattctggac aagggaatgc tggtggccat cgatgagctt 420
atgcagagcc tgaaccacaa tggcgagact cctcggcaga aacccccagt gggggaggca 480
gacccataca gggtaaagat gaagctgtgc atcctcttac atgccttttc tacgcgcgta 540
gtgaccataa acagagtgat gggcatctc tcatccgcgt gataatag 588
SEQ ID NO: 4
ggagggtcag caggaggatc tgccggtgga tcacctggtg gcagtaaa 48
SEQ ID NO: 5
aggtgtattt actagtaacg taacagtgtt acgtagtgac gtttaactga gact 54
SEQ ID NO: 6
atgggtattt actagtaacg taacagtgtt acgtagtgac gtttaactga gact 51
SEQ ID NO: 7
gctcgctttc ttgctgtcca atttctatta aaggttcctt tgttccctaa gtccaactac 60
taaactgggg gatattatga agggccttga gcatctggat tctgcctaat aaaaaacatc 120
tattttcatt gc 132
SEQ ID NO: 8
aggtgtatttactagtaacgtaacagtgttacgtagtgacgtttaa
ctgagactatgggcgtgaaggtcctgttcgccctcatttgcattg
ctgtggccgaggccatctgggaactgaagaaggatgtgtacgtgg
tggagctcgactggtatcctgatgccccgggagaaatggtggtgc
tgacgtgcgacacccccgaagaggatggtatcacctggaccctgg
atcagtcatccgaagtgctgggctcggggaaaactctgaccatcc
aagtcaaagagtttggcgacgccggacagtacacctgtcacaagg
gcggagaggtgctcagccattcgctgctcctcctgcataagaagg
aggacggcatctggtccaccgacattttgaaagaccagaaggagc
ccaagaacaagaccttcctgagatgcgaagctaagaattactccg
gccggttcacttgttggtggctgaccaccatctccactgacctga
ccttctcggtgaaaagctcaagggggtcgagcgatccgcagggcg
tgacttgtggagcagcgacgcttagcgccgaaagggtccgcggcg
acaacaaggagtacgagtactccgtggaatgccaggaggatagcg
catgtccggctgcggaagagtcgctgccgattgaagtcatggtcg
atgccgtgcacaagctgaaatacgaaaactacacctcatccttct
ttatccgggacatcattaagcctgatcctcccaagaatctgcagc
tcaagccccttaagaacagccgccaagtggaagtgtcctgggaat
acccggatacctggtccaccccgcactcctacttctccttgactt
tctgcgtccaagtgcagggaaagtccaagcgggagaagaaggacc
gggtgttcactgacaagacatccgcgaccgtgatctgccgcaaga
atgcgtccatatcagtgcgcgcccaggacagatactactcctcct
cctggtccgaatgggcctcagtcccatgctccgggggatcggccg
gtggaagcgccggtggttcgcccggaggatccaagcgcaaccttc
cggtggccactcctgacccgggaatgttcccatgtctgcaccact
cccaaaaccttctgagagcagtctcaaacatgctgcagaaggccc
ggcagactctggaattctacccctgcacctcggaggaaatcgatc
acgaggacattactaaggacaagacctctaccgtggaggcctgcc
tgcctctcgagctgaccaagaacgagagctgcctgaacagcaggg
aaacctcctttatcaccaacggaagctgcctggccagcagaaaga
cttccttcatgatggccctgtgcctgagcagcatctacgaggacc
tcaagatgtatcaggtcgagttcaagactatgaacgccaagcttc
tgatggaccccaagcggcagattttcctggaccagaacatgctgg
ccgtgattgacgaactgatgcaggccctgaacttcaactccgaaa
ccgtgccccaaaagtcgagcctggaggagccagacttctacaaga
ccaagatcaagctctgcatcctgctgcatgcgttccggatccggg
ctgtgacaattgaccgcgtgatgtcctacctcaacgcctcctgat
aataggctcgctttcttgctgtccaatttctattaaaggttcctt
tgttccctaagtccaactactaaactgggggatattatgaagggc
cttgagcatctggattctgcctaataaaaaacatttattttcatt
gcaaaaaaaaaaaaaaaaaaaaaaaaaaaaaaaaaaaaaaaaaaa
aaaaaaaaaaaaaaaaaaaaaaaaaaaaaaaaaaaaaaaaaaaaa
aaaaaaaaaaaaaaaaaaaaaaaaaaaaaaaaaaaa
SEQ ID NO: 9
atctgggaac tgcagaagga tgtgtacgtg gtggagctcg actggtatcc tgatgccccg 60
ggagaaatgg tggtgctgac gtgcgacacc cccgaagagg atggtatcac ctggaccctg 120
gatcagtcat ccgaagtgct gggctcgggg aaaactctga ccatccaagt caaagagttt 180
ggcgacgccg gacagtacac ctgtcacaag ggcggagagg tgctcagcca ttcgctgctc 240
ctcctgcata agaaggagga cggcatctgg tccaccgaca ttttgaaaga ccagaaggag 300
cccaagaaca agaccttcct gagatgcgaa gctaagaatt actccggccg gttcacttgt 360
tggtggctga ccaccatctc cactgacctg accttctcgg tgaaaagctc aagggggtcg 420
agcgatccgc agggcgtgac ttgtggagca gcgacgctta gcgccgaaag ggtccgcggc 480
gacaacaagg agtacgagta ctccgtggaa tgccaggagg atagcgcatg tccggctgcg 540
gaagagtcgc tgccgattga agtcatggtc gatgccgtgc acaagctgaa atacgaaaac 600
tacacctcat ccttctttat ccgggacatc attaagcctg atcctcccaa gaatctgcag 660
ctcaagcccc ttaagaacag ccgccaagtg gaagtgtccc gggaataccc cgatacctgg 720
tccaccccgc actcctactt ctccttgact ttctgcgtcc aagtgcaggg aaagtccaag 780
cgggagaaga aggaccgggt gttcactgac aagacatccg cgaccgtgat ctgccgcaag 840
aatgcgtcca tatcagtgca ggcccaggac agatactact cctcctcctg atccgaatgg 900
gcctcagtcc catgctcc 918
SEQ ID NO: 10
cgcaaccttc cggtggccac tcctgacccg ggaatgttcc catgtctgca ccactcccaa 60
aaccttctga gagcagtctc aaacatgctg cagaaggccc ggcagactct ggaattctac 120
ccctgcacct cggaggaaat cgatcacgag gacattacta aggacaagac ctctaccgtg 180
gaggcctgcc tgcctctcga gctgaccaag aacgagagct gcctgaacag cagggaaacc 240
tcctttatca ccaacggaag ctgcctggcc agcagaaaga cttccttcat gatggccctg 300
tgcctgagca gcatctacga ggacctcaag atgtatcagg tcgagttcaa gactatgaac 360
gccaagcttc tgatggaccc caagcggcag attttcctgg accagaacat gctggccgtg 420
attgacgaac tgatgcaggc cctgaacttc aactccgaaa ccgtgcccca aaagtcgagc 480
ctggaggagc cagacttcta gaagaccaag atcaagctct gcatcctgct gcatgcgttc 540
cggatccggg ctgtgacaat tgaccgcgtg atgtcctacc tcaacgcctc ctgataatag 600
SEQ ID NO: 11
gggggatcgg ccggtggaag cgccggtggt tcgcccggag gatccaag 48
SEQ ID NO: 12
atgggcgtga aggtcctgtt cgccctcatt tgcattgctg tggccgaggc c 51

1. An mRNA modified with 5′-cap analog, comprising the 5′-cap analog of formula (I) below or a stereoisomer, tautomer or salt thereof: 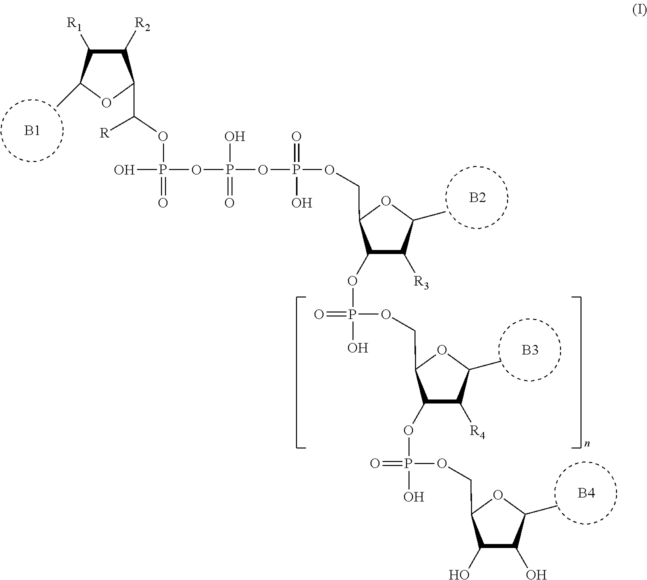
wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl; and
n is an integer selected from 0-1,
wherein said mRNA comprises, from 5′ to 3′, an interleukin 12 subunit β (IL12B) polypeptide coding sequence and an interleukin 12 subunit α (IL12A) polypeptide coding sequence.

2. The mRNA modified with 5′-cap analog of claim 1, wherein ring B1 and B4 are each independently guanine (G), and ring B2 and B3 are each independently adenine (A).

3. The mRNA modified with 5′-cap analog of claim 1, wherein R is independently selected from H and methyl.

4. The mRNA modified with 5′-cap analog of claim 1, wherein R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

5. The mRNA modified with 5′-cap analog of claim 1, wherein R₁ and R₂ are each independently selected from OH, —O(CH₂)₂OCH₃ and F.

6. The mRNA modified with 5′-cap analog of claim 1, wherein R₃ is independently selected from methoxy, F and LNA.

7. The mRNA modified with 5′-cap analog of claim 1, wherein R₄ is independently selected from F and LNA.

8. An mRNA modified with 5′-cap analog, comprising the 5′-cap analog of formula (IA) below or a stereoisomer, tautomer or salt thereof: 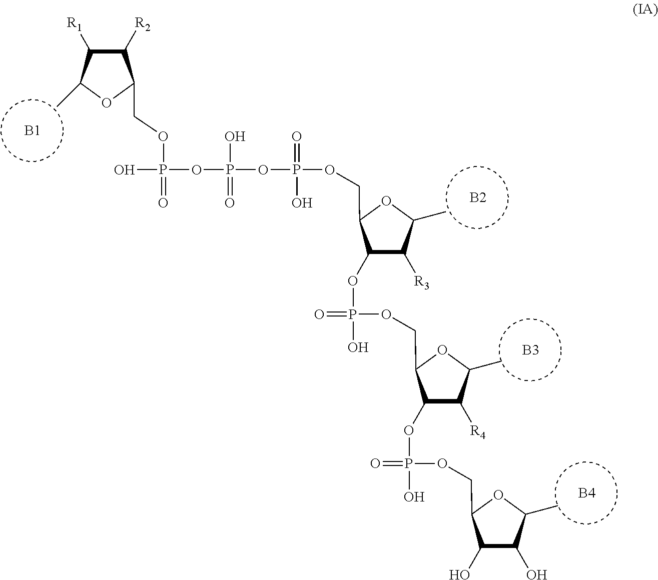
wherein:
ring B1, B2, B3 and B4 are each independently selected from a nucleobase and a modified nucleobase; and
R₃ and R₄ are each independently selected from halogen and LNA,
wherein said mRNA comprises, from 5′ to 3′, an interleukin 12 subunit β (IL12B) polypeptide coding sequence and an interleukin 12 subunit α (IL12A) polypeptide coding sequence.

9. The mRNA modified with 5′-cap analog of claim 8, wherein ring B1 and B4 are each independently guanine (G), and ring B2 and B3 are each independently adenine (A).

10. The mRNA modified with 5′-cap analog of claim 8, wherein R₃ and R₄ are each independently selected from F and LNA.

11. An mRNA modified with 5′-cap analog, comprising the 5′-cap analog of formula (IB) below or a stereoisomer, tautomer or salt thereof: 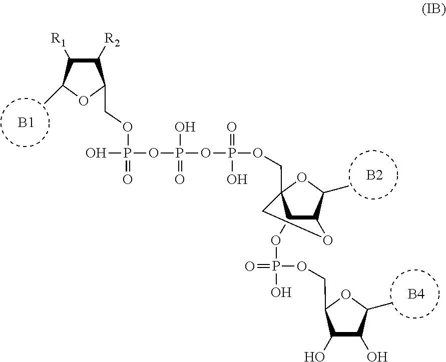
wherein:
ring B1, B2 and B4 are each independently selected from a nucleobase and a modified nucleobase;
R₁ and R₂ are each independently selected from OR₅ and halogen; and
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl,
wherein said mRNA comprises, from 5′ to 3′, an interleukin 12 subunit β (IL12B) polypeptide coding sequence and an interleukin 12 subunit α (IL12A) polypeptide coding sequence.

12. The mRNA modified with 5′-cap analog of claim 11, wherein ring B1 and B4 are each independently guanine (G), and ring B2 is independently adenine (A).

13. The mRNA modified with 5′-cap analog of claim 11, wherein R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

14. The mRNA modified with 5′-cap analog of claim 11, wherein R₁ and R₂ are each independently selected from OH, −O(CH₂)₂OCH₃ and F.

15. An mRNA modified with 5′-cap analog, comprising the 5′-cap analog selected from any of the following structures or a stereoisomer, tautomer or salt thereof: 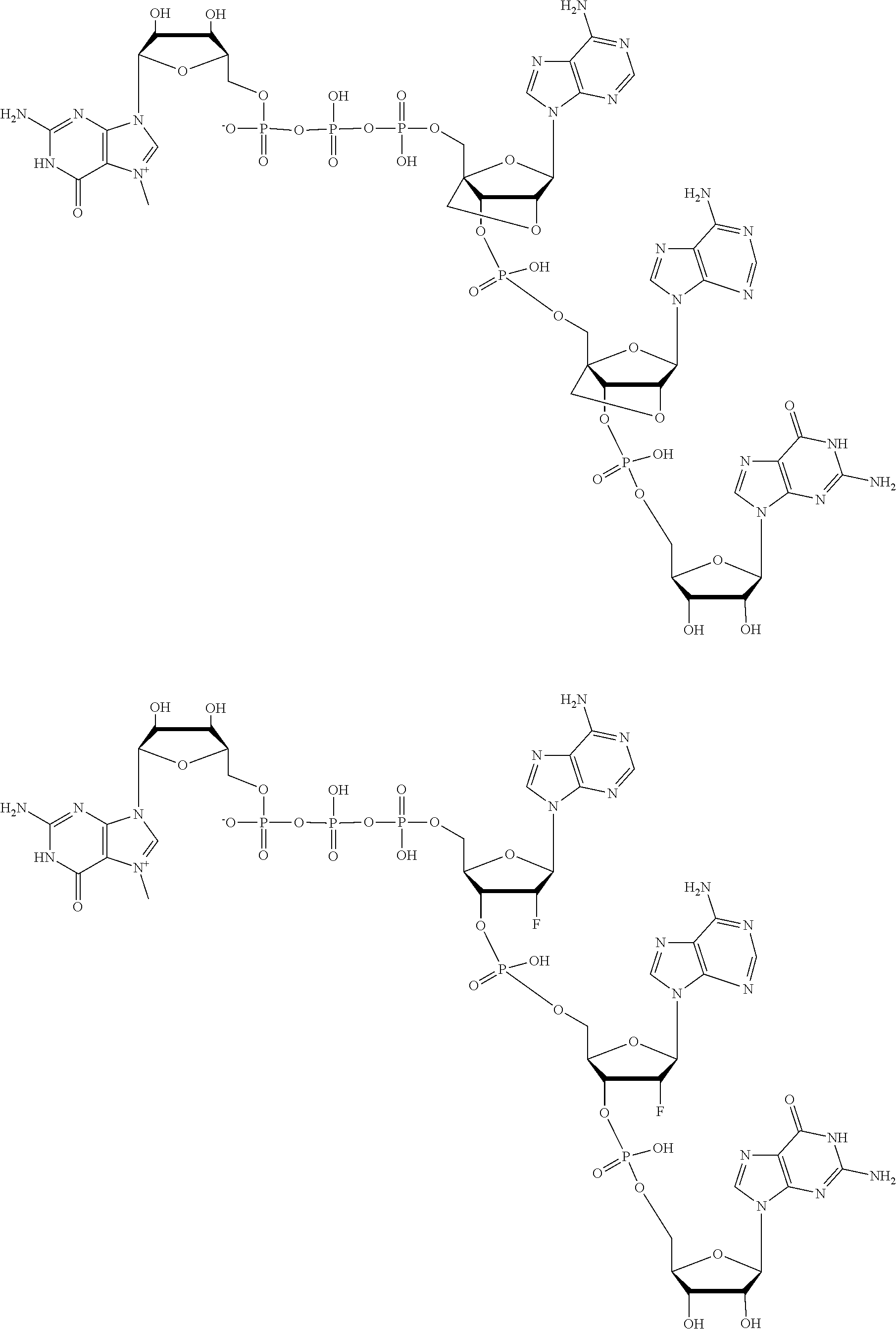 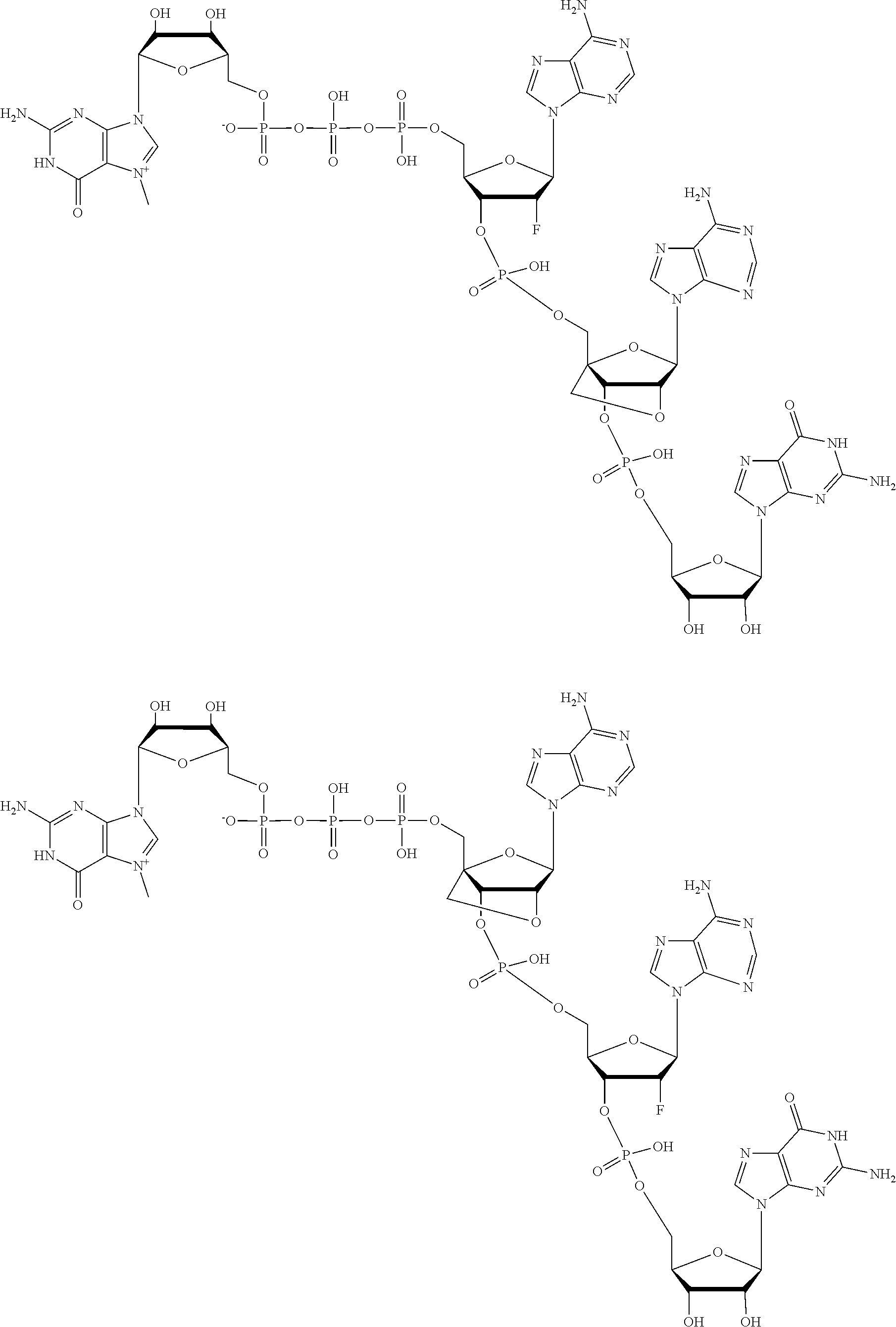 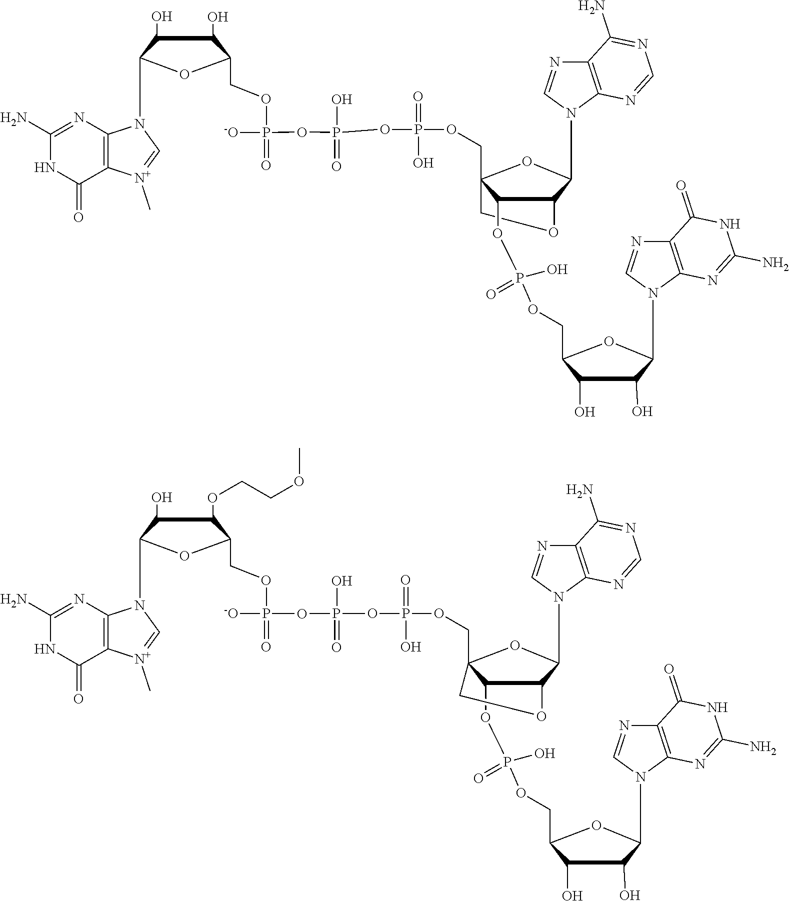 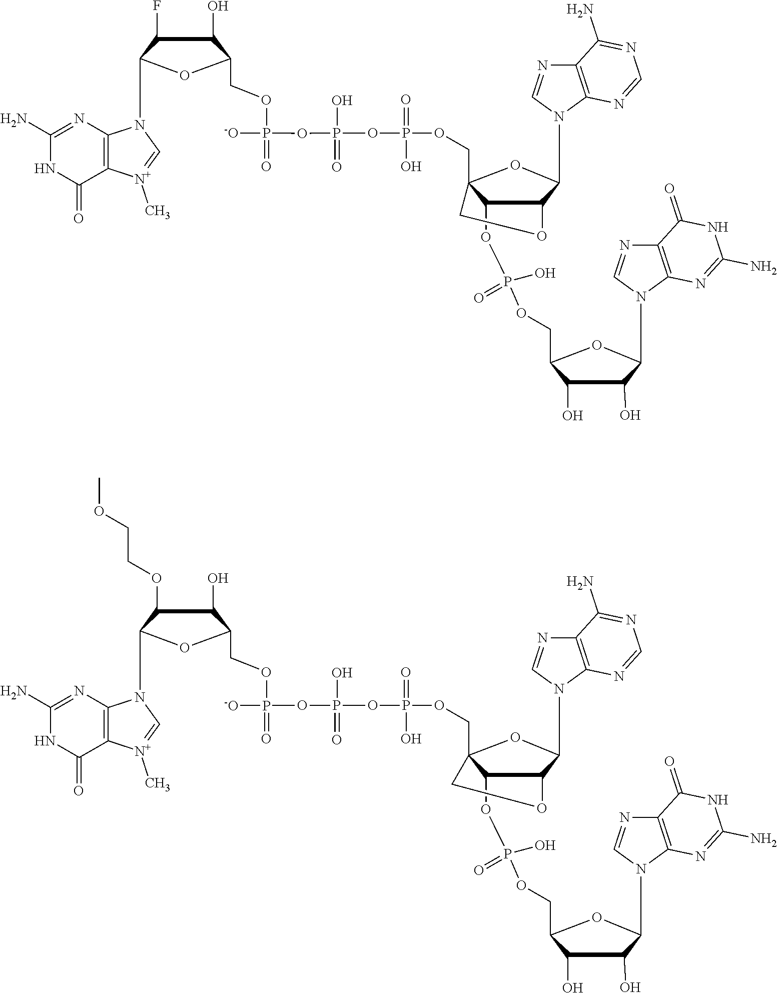 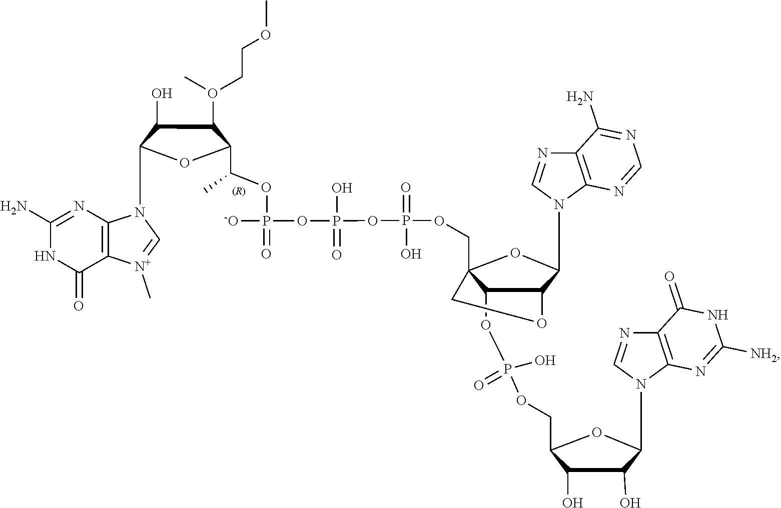
wherein said mRNA comprises, from 5′ to 3′, an interleukin 12 subunit β (IL12B) polypeptide coding sequence and an interleukin 12 subunit α (IL12A) polypeptide coding sequence.

16. The mRNA modified with 5′-cap analog of claim 1, wherein the IL12B and IL12A are human IL12B and human IL12A, or the IL12B and IL12A are mice IL12B and mice IL12A.

17. The mRNA modified with 5′-cap analog of claim 1, wherein the IL12B polypeptide coding sequence comprises SEQ ID NO:2 or SEQ ID NO:9, and/or the IL12A polypeptide coding sequence comprises SEQ ID NO:3 or SEQ ID NO:10.

18. The mRNA modified with 5′-cap analog of claim 1, wherein the mRNA further comprises a linker coding sequence linking IL12B polypeptide and IL12A polypeptide, wherein preferably said linker coding sequence comprises SEQ ID NO:4 or SEQ ID NO:11.

19. The mRNA modified with 5′-cap analog of claim 1, wherein the mRNA further comprises 5′UTR and/or 3′UTR, wherein preferably said 5′UTR comprises SEQ ID NO:5, and said 3′UTR comprises SEQ ID NO:7.

20. The mRNA modified with 5′-cap analog of claim 1, wherein the mRNA further comprises a promoter, wherein preferably said promoter comprises SEQ ID NO:6 or SEQ ID NO:12.

21. The mRNA modified with 5′-cap analog of claim 1, wherein the mRNA comprises SEQ ID NO:1 or SEQ ID NO:8.

22. A method of synthesizing the mRNA modified with 5′-cap analog of claim 1 in vitro.

23. A composition or a kit comprising the mRNA modified with 5′-cap analog of claim 1.


SEQUENCE LISTING (12 sequences)

SEQ ID NO: 1 - DNA, 1936 bp, synthetic construct
  source 1..1936: mol_type = other DNA
aggtgtatttactagtaacgtaacagtgttacgtagtgacgtttaactgagactatgggcgtaaaagttctctttgcactcatatgcattgctgtggccgaggctatgtgggaacttgaaaaagatgtgtatgtcgttgaagtggactggacgcctgacgccccgggagaaacagtgaacctcacatgtgacacaccagaggaagatgacatcacgtggacatctgatcagaggcatggcgtgattggatctgggaagactctaaccataactgttaaggaatttctggacgctgggcagtacacctgccacaaaggtggtgagaccctgagtcacagccatcttttactccataagaaagagaatggaatttggtcaactgagatcctgaagaacttcaagaacaagaccttccttaagtgtgaagcaccaaattattctggtcgcttcacatgcagctggctggtccaaaggaacatggatctgaaatttaacatcaaaagcagcagttcatctccagattccagggctgttacctgtggcatggcctctctatctgctgagaaagtgacattggaccaaagagattatgagaagtacagtgtaagctgccaggaggacgtgacttgtcccactgcagaggagacactgcctattgagctggcactggaggcgaggcaacagaacaaatatgaaaactattctactagcttttttattcgagacatcatcaagccagaccctcccaaaaatctgcaaatgaagcctctaaaaaacagtcaggtggaggtgagctgggagtaccccgattcttggagcaccccacactcctacttcagtctaaaattcttcgtccgtattcagcggaagaaggagaagatgaaagaaactgaagagggctgcaaccagaagggggcttttctcgtagaaaagacctcaactgaagtccagtgtaaaggcggcaatgtctgtgtgcaggcccaggacagatactacaattcatcatgctccaagtgggcatgtgttccctgccgagtccgtagtggagggtcagcaggaggatctgccggtggatcacctggtggcagtaaacgggtcatccctgtttcggggcctgcccgctgtctgagccaatccagaaatttgttaaaaaccacagatgacatggtgaagacggctcgagaaaagctcaagcactactcctgcacagctgaagatattgaccatgaggatataacaagagaccagaccagcacattgaagacctgcttgccgttggagctgcacaagaatgaatcttgcctggccacccgggagacttcctccactacccgcgggtcctgtcttcccccccaaaagacatccctgatgatgaccctctgtctggggagcatctatgaagatttaaaaatgtaccagacagagttccaagctatcaatgcagccctccagaatcacaaccaccagcagatcattctggacaagggaatgctggtggccatcgatgagcttatgcagagtctgaaccacaatggcgagactcttcggcagaaacccccagtgggggaggcagacccatacagggtaaagatgaagctgtgcatcctcttacatgccttttctacgcgcgtagtgaccataaacagagtgatgggctatctttcatccgcgtgataataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 939 bp, Mus musculus
  source 1..939: mol_type = other DNA
atgtgggaacttgaaaaagatgtgtatgtcgttgaagtggactggacgcctgacgccccgggagaaacagtgaacctcacatgtgacacaccagaggaagatgacatcacgtggacatctgatcagaggcatggcgtgattggatctgggaagactctaaccataactgttaaggaatttctggacgctgggcagtacacctgccacaaaggtggtgagaccctgagtcacagccatcttttactccataagaaagagaatggaatttggtcaactgagatcctgaagaacttcaagaacaagaccttccttaagtgtgaagcaccaaattattctggtcgcttcacatgcagctggctggtccaaaggaacatggatctgaaatttaacatcaaaagcagcagttcatctccagattccagggctgttacctgtggcatggcctctctatctgctgagaaagtgacattggaccaaagagattatgagaagtacagtgtaagctgccaggaggacgtgacttgtcccactgcagaggagacactgcctattgagctggcactggaggcgaggcaacagaacaaatatgaaaactattctactagcttttttattcgagacatcatcaagccagaccctcccaaaaatctgcaaatgaagcctctaaaaaacagtcaggtggaggtgagctgggagtaccccgattcttggagcaccccacactcctacttcagtctaaaattcttcgtccgtattcagcggaagaaggagaagatgaaagaaactgaagagggctgcaaccagaagggggcttttctcgtagaaaagacctcaactgaagtccagtgtaaaggcggcaatgtctgtgtgcaggcccaggacagatactacaattcatcatgctccaagtgggcatgtgttccctgccgagtccgtagt

SEQ ID NO: 3 - DNA, 588 bp, Mus musculus
  source 1..588: mol_type = other DNA
cgggtcatccctgtttcggggcctgcccgctgtctgagccaatccagaaatttgttaaaaaccacagatgacatggtgaagacggctcgagaaaagctcaagcactactcctgcacagctgaagatattgaccatgaggatataacaagagaccagaccagcacattgaagacctgcttgccgttggagctgcacaagaatgaatcttgcctggccacccgggagacttcctccactacccgcgggtcctgtcttcccccccaaaagacatccctgatgatgaccctctgtctggggagcatctatgaagatttaaaaatgtaccagacagagttccaagctatcaatgcagccctccagaatcacaaccaccagcagatcattctggacaagggaatgctggtggccatcgatgagcttatgcagagtctgaaccacaatggcgagactcttcggcagaaacccccagtgggggaggcagacccatacagggtaaagatgaagctgtgcatcctcttacatgccttttctacgcgcgtagtgaccataaacagagtgatgggctatctttcatccgcgtgataatag

SEQ ID NO: 4 - DNA, 48 bp, synthetic construct
  source 1..48: mol_type = other DNA
ggagggtcagcaggaggatctgccggtggatcacctggtggcagtaaa

SEQ ID NO: 5 - DNA, 54 bp, synthetic construct
  source 1..54: mol_type = other DNA
aggtgtatttactagtaacgtaacagtgttacgtagtgacgtttaactgagact

SEQ ID NO: 6 - DNA, 51 bp, synthetic construct
  source 1..51: mol_type = other DNA
atgggcgtaaaagttctctttgcactcatatgcattgctgtggccgaggct

SEQ ID NO: 7 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 8 - DNA, 1927 bp, synthetic construct
  source 1..1927: mol_type = other DNA
aggtgtatttactagtaacgtaacagtgttacgtagtgacgtttaactgagactatgggcgtgaaggtcctgttcgccctcatttgcattgctgtggccgaggccatctgggaactgaagaaggatgtgtacgtggtggagctcgactggtatcctgatgccccgggagaaatggtggtgctgacgtgcgacacccccgaagaggatggtatcacctggaccctggatcagtcatccgaagtgctgggctcggggaaaactctgaccatccaagtcaaagagtttggcgacgccggacagtacacctgtcacaagggcggagaggtgctcagccattcgctgctcctcctgcataagaaggaggacggcatctggtccaccgacattttgaaagaccagaaggagcccaagaacaagaccttcctgagatgcgaagctaagaattactccggccggttcacttgttggtggctgaccaccatctccactgacctgaccttctcggtgaaaagctcaagggggtcgagcgatccgcagggcgtgacttgtggagcagcgacgcttagcgccgaaagggtccgcggcgacaacaaggagtacgagtactccgtggaatgccaggaggatagcgcatgtccggctgcggaagagtcgctgccgattgaagtcatggtcgatgccgtgcacaagctgaaatacgaaaactacacctcatccttctttatccgggacatcattaagcctgatcctcccaagaatctgcagctcaagccccttaagaacagccgccaagtggaagtgtcctgggaatacccggatacctggtccaccccgcactcctacttctccttgactttctgcgtccaagtgcagggaaagtccaagcgggagaagaaggaccgggtgttcactgacaagacatccgcgaccgtgatctgccgcaagaatgcgtccatatcagtgcgcgcccaggacagatactactcctcctcctggtccgaatgggcctcagtcccatgctccgggggatcggccggtggaagcgccggtggttcgcccggaggatccaagcgcaaccttccggtggccactcctgacccgggaatgttcccatgtctgcaccactcccaaaaccttctgagagcagtctcaaacatgctgcagaaggcccggcagactctggaattctacccctgcacctcggaggaaatcgatcacgaggacattactaaggacaagacctctaccgtggaggcctgcctgcctctcgagctgaccaagaacgagagctgcctgaacagcagggaaacctcctttatcaccaacggaagctgcctggccagcagaaagacttccttcatgatggccctgtgcctgagcagcatctacgaggacctcaagatgtatcaggtcgagttcaagactatgaacgccaagcttctgatggaccccaagcggcagattttcctggaccagaacatgctggccgtgattgacgaactgatgcaggccctgaacttcaactccgaaaccgtgccccaaaagtcgagcctggaggagccagacttctacaagaccaagatcaagctctgcatcctgctgcatgcgttccggatccgggctgtgacaattgaccgcgtgatgtcctacctcaacgcctcctgataataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 918 bp, Homo sapiens
  source 1..918: mol_type = other DNA
atctgggaactgaagaaggatgtgtacgtggtggagctcgactggtatcctgatgccccgggagaaatggtggtgctgacgtgcgacacccccgaagaggatggtatcacctggaccctggatcagtcatccgaagtgctgggctcggggaaaactctgaccatccaagtcaaagagtttggcgacgccggacagtacacctgtcacaagggcggagaggtgctcagccattcgctgctcctcctgcataagaaggaggacggcatctggtccaccgacattttgaaagaccagaaggagcccaagaacaagaccttcctgagatgcgaagctaagaattactccggccggttcacttgttggtggctgaccaccatctccactgacctgaccttctcggtgaaaagctcaagggggtcgagcgatccgcagggcgtgacttgtggagcagcgacgcttagcgccgaaagggtccgcggcgacaacaaggagtacgagtactccgtggaatgccaggaggatagcgcatgtccggctgcggaagagtcgctgccgattgaagtcatggtcgatgccgtgcacaagctgaaatacgaaaactacacctcatccttctttatccgggacatcattaagcctgatcctcccaagaatctgcagctcaagccccttaagaacagccgccaagtggaagtgtcctgggaatacccggatacctggtccaccccgcactcctacttctccttgactttctgcgtccaagtgcagggaaagtccaagcgggagaagaaggaccgggtgttcactgacaagacatccgcgaccgtgatctgccgcaagaatgcgtccatatcagtgcgcgcccaggacagatactactcctcctcctggtccgaatgggcctcagtcccatgctcc

SEQ ID NO: 10 - DNA, 600 bp, Homo sapiens
  source 1..600: mol_type = other DNA
cgcaaccttccggtggccactcctgacccgggaatgttcccatgtctgcaccactcccaaaaccttctgagagcagtctcaaacatgctgcagaaggcccggcagactctggaattctacccctgcacctcggaggaaatcgatcacgaggacattactaaggacaagacctctaccgtggaggcctgcctgcctctcgagctgaccaagaacgagagctgcctgaacagcagggaaacctcctttatcaccaacggaagctgcctggccagcagaaagacttccttcatgatggccctgtgcctgagcagcatctacgaggacctcaagatgtatcaggtcgagttcaagactatgaacgccaagcttctgatggaccccaagcggcagattttcctggaccagaacatgctggccgtgattgacgaactgatgcaggccctgaacttcaactccgaaaccgtgccccaaaagtcgagcctggaggagccagacttctacaagaccaagatcaagctctgcatcctgctgcatgcgttccggatccgggctgtgacaattgaccgcgtgatgtcctacctcaacgcctcctgataatag

SEQ ID NO: 11 - DNA, 48 bp, synthetic construct
  source 1..48: mol_type = other DNA
gggggatcggccggtggaagcgccggtggttcgcccggaggatccaag

SEQ ID NO: 12 - DNA, 51 bp, synthetic construct
  source 1..51: mol_type = other DNA
atgggcgtgaaggtcctgttcgccctcatttgcattgctgtggccgaggcc